(19) United States
(12) Patent Application Publication
(10) Pub. No.: US 2022/0220095 A1
(43) Pub. Date: Jul. 14, 2022

(54) N-((HETEROARYL)METHYL)-1-TOSYL-1H-PYRAZOLE-3-CARBOXAMIDE DERIVATIVES AS Kv3 POTASSIUM CHANNEL ACTIVATORS FOR TREATING NEUROLOGICAL AND PSYCHIATRIC DISORDERS

(71) Applicant: H. Lundbeck A/S, Valby (DK)

(72) Inventors: Anette Graven SAMS, Valby (DK); Lars Kyhn RASMUSSEN, Valby (DK); Wanwan YU, Valby (DK)

(21) Appl. No.: 17/606,317

(22) PCT Filed: Apr. 24, 2020
(86) PCT No.: PCT/EP2020/061489

§ 371 Date: Oct. 25, 2021

(30) Foreign Application Priority Data
Apr. 26, 2019 (DK) PA201900487

(51) Int. Cl. C07D 403/12; C07D 413/12; C07D 401/12; C07D 417/12; C07D 405/14

(52) U.S. Cl. CPC C07D 403/12; C07D 413/12; C07D 405/14; C07D 417/12; C07D 401/12

(57) ABSTRACT

The present invention provides N-(heteroaryl)methyl)-1-tosyl-1H-pyrazole-3-carboxamide derivatives of the structure Formula (I), which activate the Kv3 potassium channels. Separate aspects of the invention are directed to pharmaceutical compositions comprising said compounds and to the compounds and pharmaceutical compositions thereof for use in methods of medical treatment of disorders responsive to the activation of Kv3 potassium channels, such as e.g. neurological or psychiatric disorders for example epilepsy, schizophrenia, cognitive impairment associated with schizophrenia (CIAS), autism spectrum disorder, bipolar disorder, ADHD, anxiety-related disorders, depression, cognitive dysfunction, Alzheimer's disease, Fragile X syndrome, chronic pain, hearing loss, sleep and circadian disorders, and sleep disruption. Exemplary compounds are e.g.: •4-methyl-N-((5-methylpyrazin-2-yl)methyl)-1-tosyl-1H-pyrazole-3-carboxamide (example 1; compound 32), •N-((1-methyl-1H-pyrazol-4-yl)methyl)-1-tosyl-1H-pyrazole-3-carboxamide (example 2; compound 62) •1-((4-(difluoro methoxy)phenypsulfonyl)-N-((5-methylpyrazin-2-yl)methyl)-1H-pyrazole-3-carboxamide (example 3; compound 65) The present description discloses the synthesis of exemplary compounds as well as relevant biological activity data (e.g. pages 24 to 47; examples 1 to 3; table 1; compounds 1 to 86).

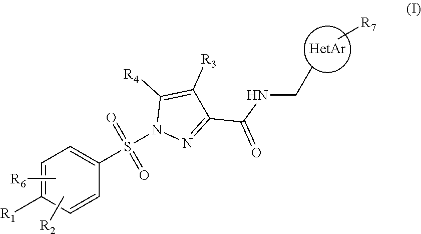

### FIELD OF THE INVENTION

The present invention relates to novel compounds which activate the Kv3 potassium channels. Separate aspects of the invention are directed to pharmaceutical compositions comprising said compounds and uses of the compounds to treat disorders responsive to the activation of Kv3 potassium channels

### BACKGROUND OF THE INVENTION

Voltage-dependent potassium (Kv) channels conduct potassium ions (K⁺) across cell membranes in response to changes in the membrane potential and can thereby regulate cellular excitability by modulating (increasing or decreasing) the electrical activity of the cell. Functional Kv channels exist as multimeric structures formed by the association of four alpha and four beta subunits. The alpha subunits comprise six transmembrane domains, a pore-forming loop and a voltage-sensor and are arranged symmetrically around a central pore. The beta or auxiliary subunits interact with the alpha subunits and can modify the properties of the channel complex to include, but not be limited to, alterations in the channel's electrophysiological or biophysical properties, expression levels or expression patterns.

Nine Kv channel alpha subunit families have been identified and are termed Kv1 through Kv9. As such, there is an enormous diversity in Kv channel function that arises as a consequence of the multiplicity of sub-families, the formation of both homomeric and heteromeric subunits within sub-families and the additional effects of association with beta subunits (Christie, 25 Clinical and Experimental Pharmacology and Physiology, 1995, 22, 944-951).

The Kv3 channel family consists of Kv3.1 (encoded by the KCNC1 gene) and Kv3.2 (encoded by the KCNC2 gene), Kv3.3 (encoded by the KCNC3 gene) and Kv3.4 (encoded by the KCNC4 gene) (Rudy and McBain, 2001). Kv3.1, Kv3.2 and Kv3.3 are prominently expressed in the central nervous system (CNS) whereas Kv3.4 expression pattern also included peripheral nervous system (PNS) and skeletal muscle (Weiser et al. 1994). Although Kv3.1, Kv3.2 and Kv3.3 channels are broadly distributed in the brain (Cerebellum, Globus pallidus, subthalamic nucleus, thalamus, auditory brain stem, cortex and hippocampus), their expression is restricted to neuronal populations able to fire action potential (AP) of brief duration and to maintain high firing rates such as fast-spiking inhibitory interneurons (Rudy and McBain, 2001). Consequently, Kv3 channels display unique biophysical properties distinguishing them from other voltage-dependent potassium channels. Kv3 channels begin to open at relatively high membrane potentials (more positive than −20 mV) and exhibit very rapid activation and deactivation kinetics (Kazmareck and Zhang; 2017). These characteristics ensure a fast repolarization and minimize the duration of after-hyperpolarization required for high frequency firing without affecting subsequent AP initiation and height.

Among Kv3 channels, Kv3.1 and Kv3.2 are particularly enriched in gabaergic interneurons including parvalbumin (PV) and somatostatin interneurons (SST) (Chow et al., 1999). Genetic ablation of Kv3.2 has been shown to broaden AP and to alter the ability to fire at high frequency in this neuronal population (Lau et al. 2000). Further, this genetic manipulation increased susceptibility to seizures. Similar phenotype was observed in mice lacking Kv3.1 and Kv3.3 confirming a crucial role of these channels in excitatory/inhibitory balance observed in epilepsy. This was confirmed at clinical level since several mutations within the KCNC1 (Kv3.1) gene have been shown to cause rare forms of epilepsy in human (Muona et al. 2015; Oliver et al. 2017). Consequently, positive modulators of Kv3 channel activators might restore excitatory/inhibitor imbalance, associated with epilepsy, through increasing the activity of inhibitory interneuron.

In addition to seizure susceptibility, excitatory/inhibitory imbalance has been postulated to participate in cognitive dysfunctions observed in a broad number of psychiatric disorders, including schizophrenia and autism spectrum disorder (Foss-Feig et al., 2017) as well as bipolar disorder, ADHD (Edden et al., 2012), anxiety-related disorders (Fuchs et al., 2017), and depression (Klempan et al., 2009). Post-mortem studies revealed alterations of the certain gabaergic molecular markers in patients suffering from these pathologies (Straub et al., 2007; Lin and Sibille, 2013). Importantly, inhibition from parvalbumin and somatostatin interneurons projecting to the pyramidal excitatory neurons is essential for the synchronized oscillatory activity of neural network, such as gamma oscillations (Bartos et al., 2007; Veit et al., 2017). This last type oscillation regulates diverse cognitive processes from sensory integration, attention, working memory and cognitive flexibility, domains that are particularly affected in psychiatric disorders (Herrmann and Demiralp; 2005). Therefore, Kv3 channel activators might rescue cognitive dysfunction and their associated alteration in gamma oscillations by increasing interneurons functions.

Both epileptiform activities and alterations of oscillations in the range of gamma have been observed at preclinical as well as clinical level in Alzheimer's disease (Palop and Mucke, 2016). While there is no current evidence of Kv3 channels alterations in Alzheimer's disease, Kv3 activators through their actions on interneurons could relieve both network alterations but also cognitive abnormalities observed in this pathology and other neurodegenerative disorders. Kv3.1 channels are particularly enriched in auditory brain stem. This particular neuronal population required to fire AP at high rated up to 600 Hz and genetic ablation of Kv3.1 alters the ability of these neurons to follow high frequency stimulation (Macica et al., 2003). Kv3.1 levels in this structure has been shown to be altered in various conditions affecting auditory sensitivity such as hearing loss (Von Hehn et al. 2004), Fragile X (Strumbos et al 2010) or tinnitus, suggesting that Kv3 activators might have therapeutic potential in these disorders.

Kv3.4 channels and to a less extent Kv3.1 are expressed in the dorsal root ganglion (Tsantoulas and McMahon 2014). Hypersensitivity to noxious stimuli in animal models of chronic pain have been associated with AP broadening (Chien et al. 2007). This phenomenon is partially due to alteration of Kv3.4 expression and function supporting the rational to use Kv3 channels activator in the treatment of certain chronic pain conditions.

Kv3.1 and Kv3.2 are widely distributed within suprachiasmic nucleus, a structure responsible for controlling circadian rhythms. Mice lacking both Kv3.1 and Kv3.2 exhibit fragmented and altered circadian rhythm (Kudo et al. 2011). Consequently, Kv3.1 channel activators might be relevant for the treatment of sleep and circadian disorders, as well as sleep disruption as core symptom of psychiatric and neurodegenerative disorders.

Autifony Therapeutics is developing AUT-00206 (AUT-6; AUT-002006), a Kv3 subfamily voltage-gated potassium channel modulator, for the potential oral treatment of schizophrenia and Fragile X. Autifony is also developing another Kv3 subfamily voltage-gated potassium channel modulator, AUT-00063, for the potential treatment of hearing disorders, including noise-induced hearing loss. The compounds are disclosed WO2017103604 and WO2018020263.

Although patients suffering from the above-mentioned disorders may have available treatment options, many of these options lack the desired efficacy and are accompanied by undesired side effects. Therefore, an unmet need exists for novel therapies for the treatment of said disorders.

In an attempt to identify new therapies, the inventors have identified a series of novel compounds as represented by Formula I which act as Kv3 channel activators, in particular as Kv3.1 channel activators. Accordingly, the present invention provides novel compounds as medicaments for the treatment of disorders which are modulated by the potassium channels.

### SUMMARY OF THE INVENTION

The present invention relates to a compound of Formula I (hereinafter also referred to as Compound (I))

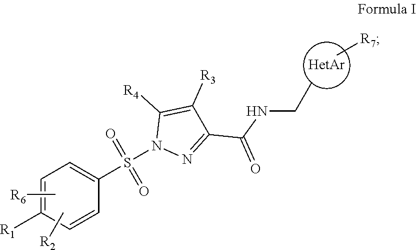

wherein
   R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl, and halogen, such as fluorine and chlorine;
   R2 and R6 are independently selected from the group consisting of H, C₁-C₄ alkyl, C₁-C₄ alkoxy, and halogen, such as fluorine and chlorine;
   R3 is selected from the group consisting of H, fluorine and C₁-C₄ alkyl;
   R4 is selected from the group consisting of H and fluorine
   R7 is selected from the group consisting of H, C₁-C₄ alkyl, halogen, such as fluorine and chlorine, C₁-C₄ alkoxy, C₁-C₄ fluoroalkyl and C₁-C₄ fluoroalkoxy;
   HetAr is selected from the group consisting of 5-membered heteroaryl and 6-membered heteroaryl;
   when R1 is C₁-C₄ alkyloxy, it may form a ring closure with R2 or R6 when any one of these are C₁-C₄ alkyl;
      or pharmaceutically acceptable salts of Compound (I).

The invention also concerns a pharmaceutical composition comprising a compound according to the invention and a pharmaceutically acceptable excipient.

Furthermore, the invention concerns Compound (I) for use as a medicament.

Further, the invention concerns use of Compound (I) for the treatment or alleviation of epilepsy, schizophrenia, in particular cognitive impairment associated with schizophrenia (CIAS), autism spectrum disorder, bipolar disorder, ADHD, anxiety-related disorders, depression, cognitive dysfunction, Alzheimer's disease, Fragile X syndrome, chronic pain, hearing loss, sleep and circadian disorders and sleep disruption.

### DETAILED DESCRIPTION OF THE INVENTION

The invention is described in further detail below, first in general and then in more detail in the embodiments of the invention and the following Experimental Section.

The present invention provides novel compounds that may be useful as medicaments for the treatment of disorders which are modulated by the potassium channels. The compounds of the invention have the generalized structure of Formula I:

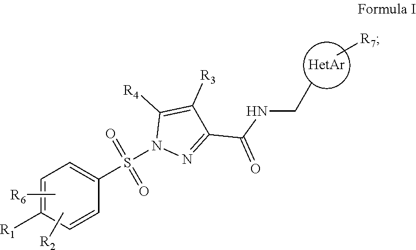

wherein R1 to R7 and HetAr are selected as disclosed above and in the more particular embodiments below.

According to a specific embodiment of the invention the compound is selected from a group of compounds as described below.

Reference to compounds encompassed by the present invention includes racemic and chiral mixtures of the compounds, optically pure isomers of the compounds for which this is relevant as well as well as tautomeric forms the compounds for which this is relevant.

Furthermore, the invention includes compounds in which one or more hydrogen has been exchanged by deuterium.

Furthermore, the compounds of the present invention may potentially exist as polymorphic and amorphic forms and in unsolvated as well as in solvated forms with pharmaceutically acceptable solvents such as water and ethanol. Both solvated and unsolvated forms of the compounds are encompassed by the present invention.

The compound according to the invention may be in a pharmaceutical composition comprising the compound and a pharmaceutically acceptable excipient.

In one embodiment, the invention relates to a compound according to the invention for use in therapy.

In another embodiment, the invention relates to a method of treating a patient in the need thereof suffering from epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile x syndrome, pain, sleep disorder and circadian disorders, comprising administering to the subject a therapeutically effective amount of a compound according to the invention.

According to an embodiment the compounds of the invention are for use as a medicament. In a particular embodiment, the compounds of the invention are for use in treating or alleviating epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile x syndrome, pain, sleep disorder and circadian disorders

In another embodiment, the compound of the invention is for the manufacture of a medicament for the treatment of epilepsy, schizophrenia, schizoaffective disorder, cognitive impairment associated with schizophrenia, bipolar disorder, ADHD, anxiety, depression, cognitive dysfunction, Alzheimer's disease, hearing loss, tinnitus, fragile x syndrome, pain, sleep disorder and circadian disorders.

The notation R1, R2, R3, R4, R6 and R7 may be used interchangeably with the notation R₁, R₂, R₃, R₄, R₆, and R₇.

A given range may interchangeably be indicated with “-” (dash) or “to”, e.g. the term “C₁₋₄ alkyl” is equivalent to “C₁ to C₄ alkyl”.

The term “C₁₋₄ alkyl” refer to an unbranched or branched saturated hydrocarbon having from one up to four carbon atoms, inclusive. Examples of such groups include, but are not limited to, methyl, ethyl, 1-propyl, 2-propyl, 1-butyl, 2-butyl and 2-methyl-2-propyl.

The term “heteroaromatic” includes tautomeric forms of the heteroaromatic compound.

The term “C₁-C₄ alkoxy” refers to a moiety of the formula —OR, wherein R indicates C₁-C₄ alkyl as defined above, wherein the alkyl part has 1, 2, 3 or 4 carbon atoms. Examples of “C₁₋₄ alkoxy” include, but are not limited to, methoxy, ethoxy, propoxy, n-butoxy and tert-butoxy.

The term “C₁₋₄ fluoroalkyl” refers to an alkyl having 1 to 4 carbon atoms, wherein at least one hydrogen atom is replaced with a fluorine atom, such as mono-, di-, or tri-fluoroalkyl.

Examples of fluoroalkyls include, but are not limited to, monofluoromethyl, difluoromethyl, trifluoromethyl, monofluoroethyl, difluoroethyl, trifluoroethyl, monofluoropropyl, difluoropropyl, trifluoropropyl, monofluorobutyl, difluorobutyl, trifluorobutyl. Preferably the fluorine atom(s) is positioned on the terminal carbon atom.

The term “C₁₋₄ fluoroalkoxy” refers to a moiety of the formula —OR_{A}, wherein R_{A} indicates C₁-C₄ fluoroalkyl as defined above. Examples of fluoroalkoxys include, but are not limited to, monofluoromethoxy, difluoromethoxy, trifluoromethoxy, monofluoroethoxy, difluoroethoxy, trifluoroethoxy, monofluoropropoxy, difluoropropoxy, trifluoropropoxy, monofluorobutoxy, difluorobutoxy, trifluorobutoxy.

The term “C₁₋₄ thioalkyl” refers to a moiety of the formula —SR, wherein R indicates C₁-C₄ alkyl as defined above. Examples of thioalkyl include, but are not limited to, thiomethyl, thioethyl, 1-thiopropyl, 2-thiopropyl, 1-thiobutyl, 2-thiobutyl and 2-methyl-2-thiopropyl.

The term “C₁₋₄ thiofluoroalkyl” refers to a moiety of the formula —SR_{A}, wherein R_{A} indicates C₁-C₄ fluoroalkyl as defined above. Examples of thiofluoroalkyls include, but are not limited to, thiomonofluoromethyl, thiodifluoromethyl, thiotrifluoromethyl, thiomonofluoroethyl, thiodifluoroethyl, thiotrifluoroethyl, thiomonofluoropropyl, thiodifluoropropyl, thiotrifluoropropyl, thiomonofluorobutyl, thiodifluorobutyl, thiotrifluorobutyl.

The term “C₃-C₈ cycloalkyl” typically refers to cyclopropyl, cyclobutyl, cyclopentyl, cyclohexyl, cycloheptyl or cyclooctyl.

The term “heteroaryl” refers to an aromatic ring or fused aromatic rings wherein one or more ring atoms are selected from O, N or S. Examples of heteroaryls include, but are not limited to, pyrimidinyl, pyridazinyl, pyrazinyl, pyrazolyl, pyridyl, oxadiazolyl, isoxazolyl, oxazolyl, thiazolyl, imidazolyl, triazolyl, thiadiazolyl and imidazopyrimidinyl.

Administration Routes

Pharmaceutical compositions comprising a compound of the present invention defined above, may be specifically formulated for administration by any suitable route such as the oral, rectal, nasal, buccal, sublingual, transdermal and parenteral (e.g. subcutaneous, intramuscular, and intravenous) route; the oral route being preferred.

It will be appreciated that the route will depend on the general condition and age of the subject to be treated, the nature of the condition to be treated and the active ingredient.

Pharmaceutical Formulations and Excipients

In the following, the term, “excipient” or “pharmaceutically acceptable excipient” refers to pharmaceutical excipients including, but not limited to, fillers, antiadherents, binders, coatings, colours, disintegrants, flavours, glidants, lubricants, preservatives, sorbents, sweeteners, solvents, vehicles and adjuvants.

The present invention also provides a pharmaceutical composition comprising a compound according to the invention, such as one of the compounds disclosed in the Experimental Section herein. The present invention also provides a process for making a pharmaceutical composition comprising a compound according to the invention. The pharmaceutical compositions according to the invention may be formulated with pharmaceutically acceptable excipients in accordance with conventional techniques such as those disclosed in Remington, “The Science and Practice of Pharmacy”, 22^{th} edition (2013), Edited by Allen, Loyd V., Jr.

In an embodiment, the present invention relates to a pharmaceutical composition comprising a compound of formula I, such as one of the compounds disclosed in the Experimental Section herein.

Pharmaceutical compositions for oral administration include solid oral dosage forms such as tablets, capsules, powders and granules; and liquid oral dosage forms such as solutions, emulsions, suspensions and syrups as well as powders and granules to be dissolved or suspended in an appropriate liquid.

Solid oral dosage forms may be presented as discrete units (e.g. tablets or hard or soft capsules), each containing a predetermined amount of the active ingredient, and preferably one or more suitable excipients. Where appropriate, the solid dosage forms may be prepared with coatings such as enteric coatings or they may be formulated so as to provide modified release of the active ingredient such as delayed or extended release according to methods well known in the art. Where appropriate, the solid dosage form may be a dosage form disintegrating in the saliva, such as for example an orodispersible tablet.

Examples of excipients suitable for solid oral formulation include, but are not limited to, microcrystalline cellulose, corn starch, lactose, mannitol, povidone, croscarmellose sodium, sucrose, cyclodextrin, talcum, gelatin, pectin, magnesium stearate, stearic acid and lower alkyl ethers of cellulose. Similarly, the solid formulation may include excipients for delayed or extended release formulations known in the art, such as glyceryl monostearate or hypromellose.

If solid material is used for oral administration, the formulation may for example be prepared by mixing the active ingredient with solid excipients and subsequently compressing the mixture in a conventional tableting machine; or the formulation may for example be placed in a hard capsule e.g. in powder, pellet or mini tablet form. The amount of solid excipient will vary widely but will typically range from about 25 mg to about 1 g per dosage unit.

Liquid oral dosage forms may be presented as for example elixirs, syrups, oral drops or a liquid filled capsule. Liquid oral dosage forms may also be presented as powders for a solution or suspension in an aqueous or non-aqueous liquid. Examples of excipients suitable for liquid oral formulation include, but are not limited to, ethanol, propylene glycol, glycerol, polyethylenglycols, poloxamers, sorbitol, poly-sorbate, mono and di-glycerides, cyclodextrins, coconut oil, palm oil, and water. Liquid oral dosage forms may for example be prepared by dissolving or suspending the active ingredient in an aqueous or non-aqueous liquid, or by incorporating the active ingredient into an oil-in-water or water-in-oil liquid emulsion.

Further excipients may be used in solid and liquid oral formulations, such as colourings, flavourings and preservatives etc.

Pharmaceutical compositions for parenteral administration include sterile aqueous and nonaqueous solutions, dispersions, suspensions or emulsions for injection or infusion, concentrates for injection or infusion as well as sterile powders to be reconstituted in sterile solutions or dispersions for injection or infusion prior to use. Examples of excipients suitable for parenteral formulation include, but are not limited to water, coconut oil, palm oil and solutions of cyclodextrins. Aqueous formulations should be suitably buffered if necessary and rendered isotonic with sufficient saline or glucose.

Other types of pharmaceutical compositions include suppositories, inhalants, creams, gels, dermal patches, implants and formulations for buccal or sublingual administration.

It is requisite that the excipients used for any pharmaceutical formulation comply with the intended route of administration and are compatible with the active ingredients.

Doses

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the gender, the age, the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of a compound of the present invention, such as 1-500 mg/day, such as 1-100 mg/day or 1-50 mg/day. Conveniently, the compounds of the invention are administered in a unit dosage form containing said compounds in an amount of about 0.1 to 500 mg, such as 10 mg, 50 mg 100 mg, 150 mg, 200 mg or 250 mg of a compound of the present invention.

Pharmaceutically Acceptable Salts

The compounds of this invention are generally utilized as the free substance or as a pharmaceutically acceptable salt thereof. When a compound of formula I contains a free base, such salts may be prepared in a conventional manner by treating a solution or suspension of a free base of formula I with a molar equivalent of a pharmaceutically acceptable acid. Representative examples of suitable organic and inorganic acids are described below.

Pharmaceutically acceptable salts in the present context is intended to indicate non-toxic, i.e. physiologically acceptable salts. The term pharmaceutically acceptable salts includes salts formed with inorganic and/or organic acids such as hydrochloric acid, hydrobromic acid, phosphoric acid, nitrous acid, sulphuric acid, benzoic acid, citric acid, gluconic acid, lactic acid, maleic acid, succinic acid, tartaric acid, acetic acid, propionic acid, oxalic acid, maleic acid, fumaric acid, glutamic acid, pyroglutamic acid, salicylic acid, salicylic acid and sulfonic acids, such as methanesulfonic acid, ethanesulfonic acid, toluenesulfonic acid and benzenesulfonic acid. Some of the acids listed above are di- or tri-acids, i.e. acids containing two or three acidic hydrogens, such as phosphoric acid, sulphuric acid, fumaric acid and maleic acid. Di- and tri-acids may form 1:1, 1:2 or 1:3 (tri-acids) salts, i.e. a salt formed between two or three molecules of the compound of the present invention and one molecule of the acid.

Additional examples of useful acids and bases to form pharmaceutically acceptable salts can be found e.g. in Stahl and Wermuth (Eds) “Handbook of Pharmaceutical salts. Properties, selection, and use”, Wiley-VCH, 2008

Isomeric and Tautomeric Forms

When compounds of the present invention contain one or more chiral centers reference to any of the compounds will, unless otherwise specified, cover the enantiomerically or diastereomerically pure compound as well as mixtures of the enantiomers or diastereomers in any ratio.

Furthermore, some of the compounds of the present invention may exist in different tautomeric forms and it is intended that any tautomeric forms that the compounds are able to form are included within the scope of the present invention.

Deuterated Compounds

Included in the scope of the present invention are also compounds of the invention in which one or more hydrogen has been exchanged by deuterium.

Therapeutically Effective Amount

In the present context, the term “therapeutically effective amount” of a compound means an amount sufficient to alleviate, arrest, partly arrest, remove or delay the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

Treatment and Treating

In the present context, “treatment” or “treating” is intended to indicate the management and care of a patient for the purpose of alleviating, arresting, partly arresting, removing or delaying progress of the clinical manifestation of the disease. The patient to be treated is preferably a mammal, in particular a human being.

All references, including publications, patent applications and patents, cited herein are hereby incorporated by reference in their entirety and to the same extent as if each reference were individually and specifically indicated to be incorporated by reference and were set forth in its entirety (to the maximum extent permitted by law).

Headings and sub-headings are used herein for convenience only, and should not be construed as limiting the invention in any way.

The use of any and all examples, or exemplary language (including “for instance”, “for example”, “e.g.”, and “as such”) in the present specification is intended merely to better illuminate the invention and does not pose a limitation on the scope of invention unless otherwise indicated.

The citation and incorporation of patent documents herein is done for convenience only, and does not reflect any view of the validity, patentability and/or enforceability of such patent documents.

The present invention includes all modifications and equivalents of the subject-matter recited in the claims appended hereto, as permitted by applicable law.

### FURTHER EMBODIMENTS OF THE INVENTION

The following embodiments describes the invention in further detail. The embodiments are numbered consecutively, starting from number 1.

### Embodiments

1. Compound (I) of Formula I

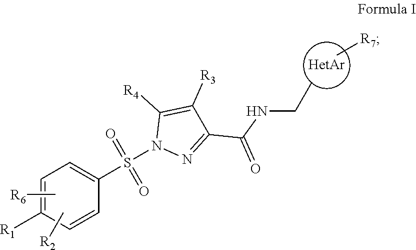

wherein
   R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl, and halogen, such as fluorine and chlorine;
   R2 and R6 are independently selected from the group consisting of H, C₁-C₄ alkyl, C₁-C₄ alkoxy, and halogen, such as fluorine and chlorine;
   R3 is selected from the group consisting of H, fluorine and C₁-C₄ alkyl;
   R4 is selected from the group consisting of H and fluorine
   R7 is selected from the group consisting of H, C₁-C₄ alkyl, halogen, such as fluorine and chlorine, C₁-C₄ alkoxy, C₁-C₄ fluoroalkyl and C₁-C₄ fluoroalkoxy;
   HetAr is selected from the group consisting of 5-membered heteroaryl and 6-membered heteroaryl;
   when R1 is C₁-C₄ alkoxy, in particular methoxy, it may form a ring closure with R2 or R6 when any one of these are C₁-C₄ alkyl, in particular methyl; or a pharmaceutically acceptable salt thereof.
2. Compound (I) according to embodiment 1, wherein
   R1 is selected from the group consisting of H, C₁-C₄ alkyl, C₁-C₄ fluoroalkyl, C₁-C₄ alkoxy, C₁-C₄ fluoroalkoxy, C₃-C₈cycloalkyl, and halogen, such as fluorine and chlorine;
   R2 and R6 are independently selected from the group consisting of H, C₁-C₄ alkyl, and halogen, such as fluorine and chlorine;
   R3 is selected from the group consisting of H, and C₁-C₄ alkyl;
   R4 is hydrogen
   R7 is selected from the group consisting of H, C₁-C₄ alkyl, halogen, such as fluorine and chlorine,
   HetAr is selected from the group consisting of 5-membered heteroaryl, 6-membered heteroaryl,
   when R1 is C₁-C₄ alkoxy, in particular methoxy, it may form a ring closure with R2 or R6 when any one of these are C₁-C₄ alkyl, in particular methyl; or a pharmaceutically acceptable salt thereof.
3. Compound (I) according to any of embodiments 1 and 2, or a pharmaceutically acceptable salt thereof, wherein R1 is selected from the group consisting of hydrogen, methyl, difluoromethyl, trifluoromethyl, trifluoromethoxy, difluoromethoxy, cyclopropyl, fluorine, chlorine and methoxy.
4. Compound (I) according to any of embodiments 1 to 3, or a pharmaceutically acceptable salt thereof, wherein R2 and R6 independently are selected from the group consisting of hydrogen, fluorine, chlorine and methyl.
5. Compound (I) according to any of embodiments 1 to 4, or a pharmaceutically acceptable salt thereof, wherein R3 is selected from the group consisting of hydrogen and methyl.
6. Compound (I) according to any of embodiments 1 to 5, or a pharmaceutically acceptable salt thereof, wherein R4 is hydrogen.
7. Compound (I) according to any of embodiments 1 to 6, or a pharmaceutically acceptable salt thereof, wherein R7 is selected from the group consisting of hydrogen, fluorine, and methyl.
8. Compound (I) according to any of embodiments 1 to 7, or a pharmaceutically acceptable salt thereof, wherein R1 is methoxy and forms a ring closure with R2 or R6 when any one of these are methyl.
9. Compound (I) according to any of embodiments 1 to 8, or a pharmaceutically acceptable salt thereof, wherein HetAr is selected from the group consisting of pyrimidinyl, pyrazinyl, pyrazolyl, pyridyl, oxadiazolyl, isoxazolyl, oxazolyl, thiazolyl, isothiazolyl, imidazolyl, triazolyl, and thiadiazolyl.
10. The compound according to any of embodiments 1 to 9, or a pharmaceutically acceptable salt thereof, selected from the group consisting of
1-(4-methoxy-3-methylbenzene-1-sulfonyl)-N-[(5-methylpyrazin-2-yl)methyl]-1H-pyrazole-3-carboxamide
1-(4-methoxy-2-methylbenzene-1-sulfonyl)-N-[(5-methylpyrazin-2-yl)methyl]-1H-pyrazole-3-carboxamide
1-(4-methoxy-2-methylbenzene-1-sulfonyl)-N-[(pyrazin-2-yl)methyl]-1H-pyrazole-3-carboxamide
1-(4-methoxy-3-methylbenzene-1-sulfonyl)-N-[(pyrazin-2-yl)methyl]-1H-pyrazole-3-carboxamide
1-(4-methoxybenzene-1-sulfonyl)-N-[(5-methylpyrazin-2-yl)methyl]-1H-pyrazole-3-carboxamide
1-(4-methoxybenzene-1-sulfonyl)-N-[(4-methyl-1,3-thiazol-2-yl)methyl]-1H-pyrazole-3-carboxamide
1-(4-methoxybenzene-1-sulfonyl)-N-[(pyrimidin-5-yl)methyl]-1H-pyrazole-3-carboxamide
1-(4-methoxybenzene-1-sulfonyl)-N-[(1,2-oxazol-3-yl)methyl]-1H-pyrazole-3-carboxamide
1-(4-methoxybenzene-1-sulfonyl)-N-[(1,2-oxazol-4-yl)methyl]-1H-pyrazole-3-carboxamide
1-(4-methoxybenzene-1-sulfonyl)-N-[(1,2-oxazol-5-yl)methyl]-1H-pyrazole-3-carboxamide
1-(4-methoxybenzene-1-sulfonyl)-N-[(2-methylpyrimidin-5-yl)methyl]-1H-pyrazole-3-carboxamide
1-(4-methoxybenzene-1-sulfonyl)-N-[(1,3-oxazol-4-yl)methyl]-1H-pyrazole-3-carboxamide
1-(4-methoxybenzene-1-sulfonyl)-N-[(1-methyl-1H-1,2,4-triazol-3-yl)methyl]-1H-pyrazole-3-carboxamide
1-(benzenesulfonyl)-N-[(3-methyl-1,2-oxazol-5-yl)methyl]-1H-pyrazole-3-carboxamide
1-(4-methyl benzene-1-sulfonyl)-N-[(5-methylpyrimidin-2-yl)methyl]-1H-pyrazole-3-carboxamide
1-(4-methyl benzene-1-sulfonyl)-N-[(5-methylpyridin-2-yl)methyl]-1H-pyrazole-3-carboxamide
1-(2-methylbenzene-1-sulfonyl)-N-[(5-methyl-1,3-oxazol-2-yl)methyl]-1H-pyrazole-3-carboxamide
1-(3-methylbenzene-1-sulfonyl)-N-[(5-methyl-1,3-oxazol-2-yl)methyl]-1H-pyrazole-3-carboxamide
1-(3-methylbenzene-1-sulfonyl)-N-[(3-methyl-1,2-oxazol-5-yl)methyl]-1H-pyrazole-3-carboxamide
1-(3-methyl benzene-1-sulfonyl)-N-[(5-methylpyridin-2-yl)methyl]-1H-pyrazole-3-carboxamide
1-(3-methylbenzene-1-sulfonyl)-N-[(1-methyl-1H-pyrazol-4-yl)methyl]-1H-pyrazole-3-carboxamide
1-(3-methylbenzene-1-sulfonyl)-N-[(1-methyl-1H-pyrazol-3-yl)methyl]-1H-pyrazole-3-carboxamide
1-(2-methylbenzene-1-sulfonyl)-N-[(3-methyl-1,2,4-oxadiazol-5-yl)methyl]-1H-pyrazole-3-carboxamide
1-(4-methylbenzene-1-sulfonyl)-N-[(pyrazin-2-yl)methyl]-1H-pyrazole-3-carboxamide
1-(2-methylbenzene-1-sulfonyl)-N-[(5-methylpyrimidin-2-yl)methyl]-1H-pyrazole-3-carboxamide
1-(2-methylbenzene-1-sulfonyl)-N-[(1-methyl-1H-pyrazol-3-yl)methyl]-1H-pyrazole-3-carboxamide
1-(2-methyl benzene-1-sulfonyl)-N-[(5-methylpyridin-2-yl)methyl]-1H-pyrazole-3-carboxamide
1-(2-methylbenzene-1-sulfonyl)-N-[(1-methyl-1H-pyrazol-4-yl)methyl]-1H-pyrazole-3-carboxamide
1-(2-methylbenzene-1-sulfonyl)-N-[(3-methyl-1,2-oxazol-5-yl)methyl]-1H-pyrazole-3-carboxamide
1-(4-methylbenzene-1-sulfonyl)-N-[(5-methyl-1,3-oxazol-2-yl)methyl]-1H-pyrazole-3-carboxamide
1-(4-fluorobenzene-1-sulfonyl)-N-[(3-methyl-1,2-oxazol-5-yl)methyl]-1H-pyrazole-3-carboxamide
4-methyl-1-(4-methylbenzene-1-sulfonyl)-N-[(5-methylpyrazin-2-yl)methyl]-1H-pyrazole-3-carboxamide
1-(4-methylbenzene-1-sulfonyl)-N-[(1,2-thiazol-4-yl)methyl]-1H-pyrazole-3-carboxamide
1-(4-fluorobenzene-1-sulfonyl)-N-[(5-methylpyridin-2-yl)methyl]-1H-pyrazole-3-carboxamide
1-(4-fluorobenzene-1-sulfonyl)-N-[(1-methyl-1H-pyrazol-3-yl)methyl]-1H-pyrazole-3-carboxamide
1-(4-methoxybenzene-1-sulfonyl)-N-[(5-methyl-1,3-oxazol-2-yl)methyl]-1H-pyrazole-3-carboxamide
1-(4-methoxybenzene-1-sulfonyl)-N-[(5-methylpyrimidin-2-yl)methyl]-1H-pyrazole-3-carboxamide
1-(4-methoxybenzene-1-sulfonyl)-N-[(3-methyl-1,2-oxazol-5-yl)methyl]-1H-pyrazole-3-carboxamide
1-(4-methylbenzene-1-sulfonyl)-N-[(1-methyl-1H-pyrazol-3-yl)methyl]-1H-pyrazole-3-carboxamide
1-(4-methoxybenzene-1-sulfonyl)-N-[(5-methyl pyridin-2-yl)methyl]-1H-pyrazole-3-carboxamide
1-(4-methoxybenzene-1-sulfonyl)-N-[(1-methyl-1H-pyrazol-4-yl)methyl]-1H-pyrazole-3-carboxamide
1-(4-methoxybenzene-1-sulfonyl)-N-[(3-methyl-1,2,4-oxadiazol-5-yl)methyl]-1H-pyrazole-3-carboxamide
1-(4-methoxybenzene-1-sulfonyl)-N-[(pyrazin-2-yl)methyl]-1H-pyrazole-3-carboxamide
1-(4-methoxybenzene-1-sulfonyl)-N-[(1-methyl-1H-pyrazol-3-yl)methyl]-1H-pyrazole-3-carboxamide
1-(4-methylbenzene-1-sulfonyl)-N-[(1,3-oxazol-2-yl)methyl]-1H-pyrazole-3-carboxamide
1-(4-methylbenzene-1-sulfonyl)-N-[(1-methyl-1H-1,2,3-triazol-4-yl)methyl]-1H-pyrazole-3-carboxamide
1-(4-methylbenzene-1-sulfonyl)-N-[(1,3,4-thiadiazol-2-yl)methyl]-1H-pyrazole-3-carboxamide
1-(4-methylbenzene-1-sulfonyl)-N-[(3-methyl-1,2,4-oxadiazol-5-yl)methyl]-1H-pyrazole-3-carboxamide
1-(4-methylbenzene-1-sulfonyl)-N-[(1,3-oxazol-4-yl)methyl]-1H-pyrazole-3-carboxamide
1-(4-methylbenzene-1-sulfonyl)-N-[(2-methylpyrimidin-5-yl)methyl]-1H-pyrazole-3-carboxamide
1-(4-methylbenzene-1-sulfonyl)-N-[(1,2-oxazol-4-yl)methyl]-1H-pyrazole-3-carboxamide
1-(4-methylbenzene-1-sulfonyl)-N-[(1,3-thiazol-4-yl)methyl]-1H-pyrazole-3-carboxamide
1-(4-methylbenzene-1-sulfonyl)-N-[(1-methyl-1H-imidazol-4-yl)methyl]-1H-pyrazole-3-carboxamide
1-(4-methylbenzene-1-sulfonyl)-N-[(pyrimidin-5-yl)methyl]-1H-pyrazole-3-carboxamide
1-(4-methylbenzene-1-sulfonyl)-N-[(1,2-oxazol-5-yl)methyl]-1H-pyrazole-3-carboxamide
1-(4-methylbenzene-1-sulfonyl)-N-[(3-methyl-1,2-oxazol-5-yl)methyl]-1H-pyrazole-3-carboxamide
1-(4-methylbenzene-1-sulfonyl)-N-[(1,2,4-oxadiazol-3-yl)methyl]-1H-pyrazole-3-carboxamide
1-(4-methylbenzene-1-sulfonyl)-N-[(1,2-oxazol-3-yl)methyl]-1H-pyrazole-3-carboxamide
1-(4-methylbenzene-1-sulfonyl)-N-[(1,3-thiazol-2-yl)methyl]-1H-pyrazole-3-carboxamide
1-(4-methylbenzene-1-sulfonyl)-N-[(1-methyl-1H-1,2,4-triazol-3-yl)methyl]-1H-pyrazole-3-carboxamide
1-(4-methylbenzene-1-sulfonyl)-N-[(4-methyl-1,3-thiazol-2-yl)methyl]-1H-pyrazole-3-carboxamide
1-(4-methylbenzene-1-sulfonyl)-N-[(1-methyl-1H-pyrazol-4-yl)methyl]-1H-pyrazole-3-carboxamide
1-(4-methylbenzene-1-sulfonyl)-N-[(5-methylpyrazin-2-yl)methyl]-1H-pyrazole-3-carboxamide
1-(4-methoxyphenyl)sulfonyl-N-(3-pyridylmethyl)pyrazole-3-carboxamide
1-[4-(difluoromethoxy)phenyl]sulfonyl-N-[(5-methylpyrazin-2-yl)methyl]pyrazole-3-carboxamide
1-(4-fluoro-2-methyl-phenyl)sulfonyl-N-[(5-methylpyrazin-2-yl)methyl]pyrazole-3-carboxamide
1-(4-chloro-2-methyl-phenypsulfonyl-N-[(5-methylpyrazin-2-yl)methyl]pyrazole-3-carboxamide
1-(2-fluoro-4-methoxy-phenypsulfonyl-N-[(5-methylpyrazin-2-yl)methyl]pyrazole-3-carboxamide
1-(3-chloro-4-fluoro-phenyl)sulfonyl-N-[(5-methylpyrazin-2-yl)methyl]pyrazole-3-carboxamide
N-[(5-methylpyrazin-2-yl)methyl]-1-[4-(trifluoromethoxy)phenyl]sulfonyl-pyrazole-3-carboxamide
1-[4-(difluoromethyl)phenyl]sulfonyl-N-[(5-methylpyrazin-2-yl)methyl]pyrazole-3-carboxamide
1-(2,3-dihydrobenzofuran-5-ylsulfonyl)-N-[(5-methylpyrazin-2-yl)methyl]pyrazole-3-carboxamide
1-(4-chloro-2-fluoro-phenyl)sulfonyl-N-[(5-methylpyrazin-2-yl)methyl]pyrazole-3-carboxamide
1-(3-fluoro-4-methoxy-phenypsulfonyl-N-[(5-methylpyrazin-2-yl)methyl]pyrazole-3-carboxamide
1-(3-chlorophenyl)sulfonyl-N-[(5-methylpyrazin-2-yl)methyl]pyrazole-3-carboxamide
1-(4-chlorophenyl)sulfonyl-N-[(5-methylpyrazin-2-yl)methyl]pyrazole-3-carboxamide
1-(2,4-dimethylphenyl)sulfonyl-N-[(5-methylpyrazin-2-yl)methyl]pyrazole-3-carboxamide
N-[(5-methylpyrazin-2-yl)methyl]-1-[4-(trifluoromethyl)phenyl]sulfonyl-pyrazole-3-carboxamide
1-(4-cyclopropylphenyl)sulfonyl-N-[(5-methylpyrazin-2-yl)methyl]pyrazole-3-carboxamide
1-(2-fluoro-4-methyl-phenyl)sulfonyl-N-[(5-methylpyrazin-2-yl)methyl]pyrazole-3-carboxamide
1-(4-fluoro-3,5-dimethyl-phenypsulfonyl-N-[(5-methylpyrazin-2-yl)methyl]pyrazole-3-carboxamide
1-(2-chloro-4-methoxy-phenyl)sulfonyl-N-[(5-methylpyrazin-2-yl)methyl]pyrazole-3-carboxamide
1-(4-fluoro-2,6-dimethyl-phenypsulfonyl-N-[(5-methylpyrazin-2-yl)methyl]pyrazole-3-carboxamide
1-(2-chlorophenyl)sulfonyl-N-[(5-methylpyrazin-2-yl)methyl]pyrazole-3-carboxamide
N-[(3-fluoro-2-pyridyl)methyl]-1-(4-methoxyphenyl)sulfonyl-pyrazole-3-carboxamide
11. A pharmaceutical composition comprising Compound (I) of any of embodiments 1-10, or a pharmaceutically acceptable salt thereof, and one or more pharmaceutically acceptable excipients.
12. Compound (I) of any of embodiments 1-10, or a pharmaceutically acceptable salt thereof, or the pharmaceutical composition of embodiment 11 for use in therapy.
13. Compound (I) of any of embodiments 1-10, or a pharmaceutically acceptable salt thereof, or the pharmaceutical composition of embodiment 11 for use in a method for the treatment of a neurological or psychiatric disorder.
14. A method for the treatment of a neurological or psychiatric disorder comprising the administration of a therapeutically effective amount of Compound (I) of any of embodiments 1-10, or a pharmaceutically acceptable salt thereof, or the pharmaceutical composition of embodiment 11 to a patient in need thereof.
15. Use of Compound (I) of any of embodiments 1-10, or a pharmaceutically acceptable salt thereof, or the pharmaceutical composition of embodiment 11, for the manufacture of a medicament for the treatment of a neurological or psychiatric disorder.
16. Compound (I) of any of embodiments 1-10, or a pharmaceutically acceptable salt thereof, for use in a method for the treatment of a neurological or psychiatric disorder, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, Fragile X syndrome, chronic pain, hearing loss, sleep and circadian disorders and sleep disruption.
17. The pharmaceutical composition of embodiment 11 for the use specified in embodiment 13, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, Fragile X syndrome, chronic pain, hearing loss, sleep and circadian disorders and sleep disruption.
18. Use of Compound (I) of any of embodiments 1-10, or a pharmaceutically acceptable salt thereof, for the manufacture of a medicament for the treatment of a neurological or psychiatric disorder, wherein the neurological or psychiatric disorder is selected from the group consisting of epilepsy, schizophrenia, for example of the paranoid, disorganized, catatonic, undifferentiated, or residual type; schizophreniform disorder; schizoaffective disorder, for example of the delusional type or the depressive type, cognitive impairment associated with schizophrenia (CIAS), autism spectrum disorder, bipolar disorder, ADHD, anxiety-related disorders, depression, cognitive dysfunction, Alzheimer's disease, Fragile X syndrome, chronic pain, hearing loss, sleep and circadian disorders and sleep disruption.

All references, including publications, patent applications and patents, cited herein are hereby incorporated by reference in their entirety and to the same extent as if each reference were individually and specifically indicated to be incorporated by reference and were set forth in its entirety (to the maximum extent permitted by law).

Headings and sub-headings are used herein for convenience only and should not be construed as limiting the invention in any way.

The description herein of any aspect or aspect of the invention using terms such as “comprising”, “having,” “including” or “containing” with reference to an element or elements is intended to provide support for a similar aspect or aspect of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (including “for instance”, “for example”, “e.g.”, and “as such”) in the present specification is intended merely to better illuminate the invention and does not pose a limitation on the scope of invention unless otherwise indicated.

It should be understood that the various aspects, embodiments, implementations and features of the invention mentioned herein may be claimed separately, or in any combination.

The present invention includes all modifications and equivalents of the subject-matter recited in the claims appended hereto, as permitted by applicable law.

The invention is further described by the following experimental section.

### EXPERIMENTAL SECTION

The compounds of Formula I may be prepared by methods described below, together with synthetic methods known in the art of organic chemistry, or modifications that are familiar to those skilled in the art. The starting materials used herein are available commercially or may be prepared by routine methods known in the art, such as those methods described in standard reference books such as “Compendium of Organic Synthetic Methods, Vol. I-XII” (published with Wiley-Interscience). Preferred methods include, but are not limited to, those described below.

The schemes are representative of methods useful in synthesizing the compounds of the present invention. They are not to constrain the scope of the invention in any way.

Analytical Methods

Chromatographic systems and methods to evaluate chemical purity (LCMS methods) are described below:

Method A: Apparatus: Agilent 1200 LCMS System with ELS Detector.

| Column | Waters Xbridge-C18, 50 × 2 mm, 5 μm | | |
|---|---|---|---|
| Flow rate | 0.8 mL/min | | |
| Run time | 4.5 min. | | |
| Wavelength | 254 nm | | |
| Oven temp | 50° C. | | |
| Ion source | ESI | | |
| Solvent A | Water + 0.04% TFA | | |
| Solvent B | CH₃CN (MeCN) + 0.02% TFA | | |
| Gradient | Time | A % | B % |
| | 0 | 99 | 1 |
| | 3.4 | 0 | 100 |
| | 4 | 99 | 1 |
| | 4.5 | 99 | 1 |

Method B: Apparatus: Agilent 1200 LCMS System with ELS Detector.

| Column | Waters XBridge ShieldRP18, 2.1 * 50 mm, 5 μm | | |
|---|---|---|---|
| Flow rate | 0.8 mL/min | | |
| Run time | 4.5 min. | | |
| Wavelength | 254 nm | | |
| Oven temp | 40° C. | | |
| Ion source | ESI | | |
| Solvent A | Water + 0.05% NH₃ · H₂O | | |
| Solvent B | CH₃CN (MeCN) | | |
| Gradient | Time | A % | B % |
| | 0 | 95 | 5 |
| | 3.4 | 0 | 100 |
| | 4 | 0 | 100 |
| | 4.5 | 95 | 5 |

Method D: Waters Aquity UPLC with TQD MS-Detector

| Column | Aquity UPLC BEH C18, 2.1 * 50 mm, 1.7 μm | | |
|---|---|---|---|
| Flow rate | 1.2 mL/min | | |
| Run time | 1.15 min. | | |
| Wavelength | 254 nm | | |
| Oven temp | 60° C. | | |
| Ion source | APPI | | |
| Solvent A | Water + 0.05% TFA | | |
| Solvent B | 0.035% TFA in CH₃CN 95% + Water 5% | | |
| Gradient | Time | A % | B % |
| | 0 | 90 | 10 |
| | 1 | 0 | 100 |
| | 1.15 | 90 | 10 |

Following separation by chromatography the compounds were analysed by use of ¹H NMR and/or LCMS. ¹H NMR spectra were recorded at 400.13 MHz on a Bruker Avance III 400 instrument, at 300.13 MHz on a Bruker Avance 300 instrument or at 600.16 MHz on a 600 MHz Bruker Avance III HD. Deuterated dimethyl sulfoxide or deuterated chloroform was used as solvent. Tetramethylsilane was used as internal reference standard.

Chemical shift values are expressed in ppm-values relative to tetramethylsilane. The following abbreviations are used for multiplicity of NMR signals: s=singlet, d=doublet, t=triplet, q=quartet, qui=quintet, h=heptet, dd=double doublet, dt=double triplet, dq=double quartet, tt=triplet of triplets, m=multiplet and brs=broad singlet.

### Synthesis of Compounds of the Invention

### General Methods

### Method 1

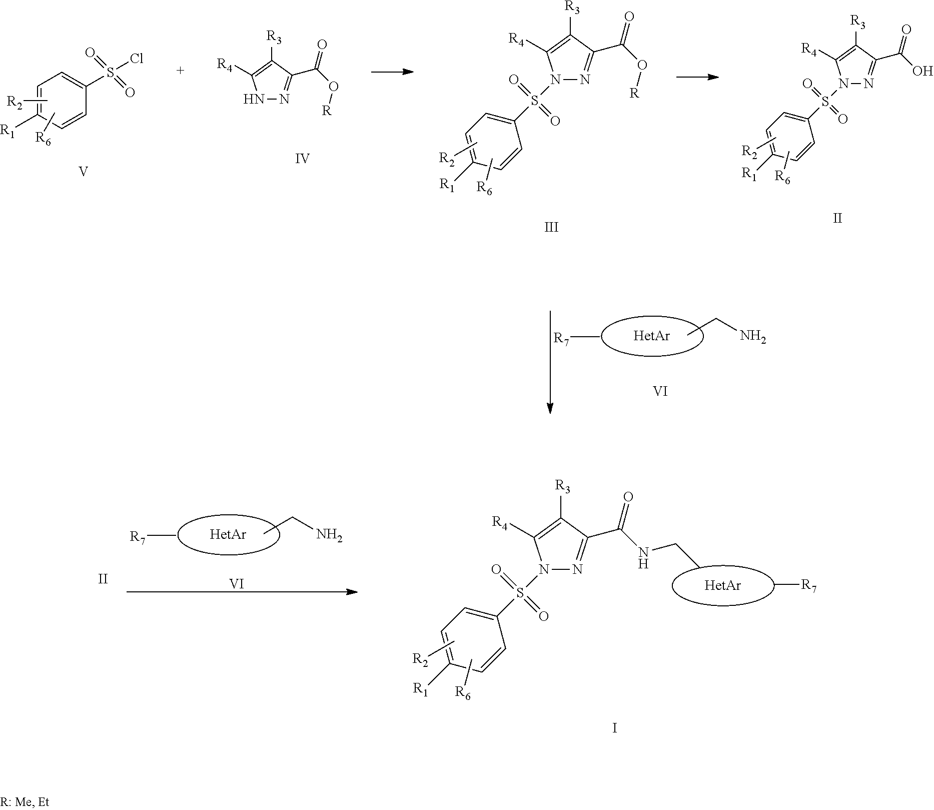

The compounds of the invention can be prepared by reacting a suitably substituted alkyl 1H-pyrazole-3-carboxylate (IV) with an arylsulfonic acid derivative exemplified by but not limited to an arylsulfonylchloride (V) in a solvent such as tetrahydrofuran, in the presence of a base exemplified by, but not limited to sodium hydride, to form intermediate III. Intermediate II can be prepared from III under standard ester hydrolysis conditions, exemplified by but not limited to aqueous lithium hydroxide in tetrahydrofuran. Compound I is formed from intermediate II by coupling with an amine (VI) under standard amide formation conditions, using a coupling reagent, such as hexafluorophosphate azabenzotriazole tetramethyl uronium (HATU), and a base exemplified by but not limited to triethylamine, in a solvent exemplified by but not limited to dichloromethane. Alternatively, compounds of the of the formula I can be prepared directly by reaction of III with a metal amide, exemplified by, but not limited to dimethylaluminium amide, in a solvent such as dichloromethane.

### Method 2

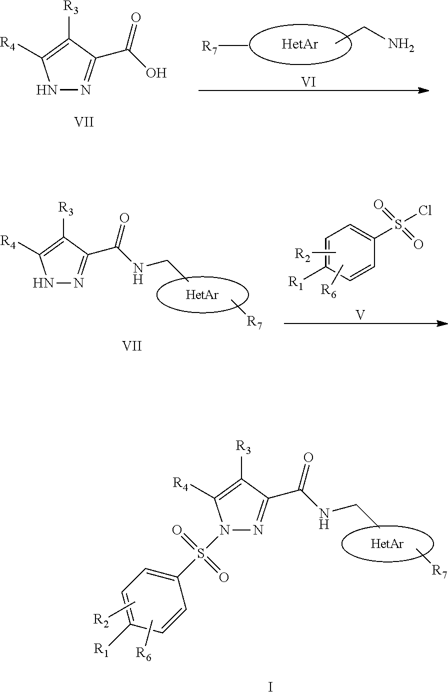

Compounds of the invention can be prepared by reacting suitably substituted 1H-pyrazole-3-carboxylic acid (VIII) with an amine under standard amide formation conditions, using a coupling reagent, such as carbonyldiimidazole (CDI) in a solvent exemplified by but not limited to tetrahydrofurane (THF). Compound I is formed by reaction of intermediate VII with an arylsulfonylchloride (V) in a solvent such as tetrahydrofuran, in the presence of a base exemplified by, but not limited to sodium hydride.

### Example 1

### Preparation of ethyl 4-methyl-1-tosyl-1H-pyrazole-3-carboxylate

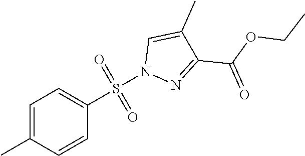 [SMILES: CCOC(=O)c1nn(S(=O)(=O)c2ccc(C)cc2)cc1C]

To a solution of ethyl 4-methylpyrazole-3-carboxylate (0.31 g, 2.0 mmol) and triethylamine (3.4 g, 34 mmol) in DMF (2 mL) was added 4-methylbenzene-1-sulfonyl chloride (0.42 g, 2.2 mmol) portionwise at room temperature. The mixture was stirred at 25° C. for 4 hours. The reaction was concentrated in vacuo and H₂O (25 mL) was added. The mixture was extracted with ethyl acetate (3×25 mL). The combined organic phases were washed with brine, dried over MgSO₄ and concentrated in vacuo. The crude material was purified by flash chromatography on silica gel using ethyl acetate and heptane to yield 0.53 g of ethyl 4-methyl-1-tosyl-1H-pyrazole-3-carboxylate.

¹H NMR (500 MHz, Chloroform-d) δ 7.93 (d, 2H), 7.89 (d, 1H), 7.36-7.32 (m, 2H), 4.37 (q, 2H), 2.43 (s, 3H), 2.25 (d, 3H), 1.37 (t, 3H).

### Preparation of 4-methyl-1-tosyl-1H-pyrazole-3-carboxylic Acid

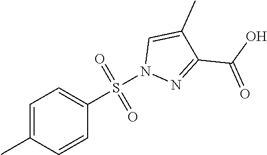 [SMILES: Cc1ccc(S(=O)(=O)n2cc(C)c(C(=O)O)n2)cc1]

Lithium hydroxide hydrate (0.41 g, 9.6 mmol) was added to ethyl 4-methyl-1-tosyl-1H-pyrazole-3-carboxylate (0.3 g, 0.96 mmol) in water (1 mL) and THF (5 mL). The mixture was stirred at 0° C. for 5 hours. The mixture was acidified by 2 M HCl (aq) (4.8 ml, 9.6 mmol) and extracted with ethyl acetate (3×50 mL). The combined organic phases were washed with brine, dried over MgSO₄ and concentrated in vacuo. Used in the next step without further purification.

### Preparation of 4-methyl-N-((5-methylpyrazin-2-yl)methyl)-1-tosyl-1H-pyrazole-3-carboxamide (Compound 32)

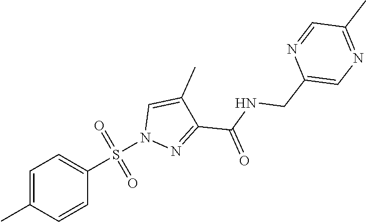

To a solution of 4-methyl-1-tosyl-1H-pyrazole-3-carboxylic acid (0.27 g, 0.96 mmol) in DCM (10 mL) and DMF (1 mL) were added HATU (0.44 g, 1.16 mmol), 1-Hydroxy-7-azabenzotriazole (0.039 g, 0.29 mmol) and DIPEA (1.87 g, 14.5 mmol). After stirring at room temperature (RT) for 30 min, (5-methylpyrazin-2-yl)methanamine (0.12 g, 0.96 mmol) was added and the mixture was stirred at room temperature for 18 hrs. The crude mixture was diluted with DCM (75 mL) and washed with sat. aq. NaHCO₃ and brine. The solution was dried over MgSO₄ and concentrated in vacuo. The crude material was purified by flash chromatography on silica gel using ethyl acetate and heptane to yield 90 mg of 4-methyl-N-((5-methylpyrazin-2-yl)methyl)-1-tosyl-1H-pyrazole-3-carboxamide.

¹H NMR (600 MHz, Chloroform-d) δ 8.49 (d, 1H), 8.39 (d, 1H), 7.85 (dd, 2H), 7.84 (s, 1H), 7.63 (t, 1H), 7.34-7.31 (m, 2H), 4.65 (d, 2H), 2.54 (s, 3H), 2.42 (s, 3H), 2.29 (d, 3H). LC-MS method D: t_{R}=0.66 min, m/z: 386.1 [M+H]⁺

Compound 1-31, 33-60 and 63 were prepared analogously from the corresponding starting materials

### Example 2: Preparation of N-((1-methyl-1H-pyrazol-4-yl)methyl)-1-tosyl-1H-pyrazole-3-carboxamide (Compound 62)

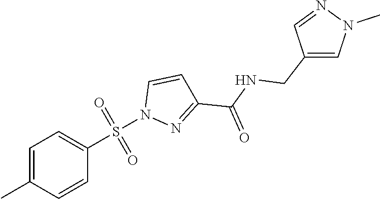

To a mixture of methyl 1-tosyl-1H-pyrazole-3-carboxylate (0.10 g, 0.36 mmol) and (1-methyl-1H-pyrazol-4-yl)methanamine dihydrochloride (40 mg, 0.36 mmol) in toluene (5 mL) was added 2 M AlMe₃ (0.54 mmol, in toluene) dropwise at 20° C. under an atmosphere of nitrogen. The mixture was stirred at 50° C. for 3 hrs. The reaction mixture was quenched by addition of sat. NH₄Cl solution (10 mL) at 0° C. The residue was poured into ice-water (30 mL) and stirred for 3 min. The aqueous phase was extracted with ethyl acetate (3×40 mL). The combined organic phases were washed with brine (2×60 mL), dried with anhydrous Na₂SO₄, filtered and concentrated. The residue was purified by preparative HPLC to afford 63 mg of N-((1-methyl-1H-pyrazol-4-yl)methyl)-1-tosyl-1H-pyrazole-3-carboxamide.

¹H NMR (DMSO-d⁶ 400 MHz): δ 8.77-8.75 (m, 1H), 8.55 (d, 1H), 7.92 (d, 2H), 7.55 (s, 1H), 7.49 (d, 2H), 7.31 (s, 1H), 6.90 (d, 1H), 4.20 (d, 2H), 3.76 (s, 3H), 2.39 (s, 3H).

LC-MS method B: t_{R}=1.716 min, m/z=359.9 [M+H]⁺.

Compound 61 was prepared analogously from the corresponding starting materials.

### Example 3

### Preparation of N-[(5-methylpyrazin-2-yl)methyl]-1H-pyrazole-3-carboxamide

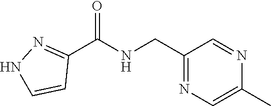 [SMILES: Cc1cnc(CNC(=O)c2cc[nH]n2)cn1]

To a solution of 1H-pyrazole-5-carboxylic acid (3.0 g, 26.8 mmol) in THF (30 mL) was slowly added portionwise carbonyldiimidazole (4.77 g, 29.4 mmol). The mixture was heated at 50° C. for 3 hrs (until evolution of gas was completed). Then 2-(aminomethyl)-5-methylpyrazine (3.63 g, 29.4 mmol) was added and the reaction mixture was stirred overnight at ambient temperature. The reaction mixture was diluted with water (100 mL) and extracted with chloroform (200 mL). The organic layer was separated, dried over sodium sulphate, filtered, and the filtrate was evaporated to give a N-[(5-methylpyrazin-2-yl)methyl]-1H-pyrazole-3-carboxamide (4.7 g) the obtained material was used without further purification.

### Preparation of 1-((4-(difluoromethoxy)phenyl)sulfonyl)-N-((5-methylpyrazin-2-yl)methyl)-1H-pyrazole-3-carboxamide (Compound 65)

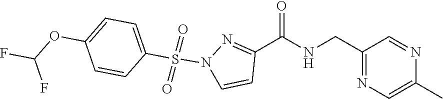 [SMILES: Cc1cnc(CNC(=O)c2ccn(S(=O)(=O)c3ccc(OC(F)F)cc3)n2)cn1]

A solution of t-BuONa (14.9 mg, 0.155 mmol, 1.2 equiv) in THF (0.5 mL) was added to a suspension of N-((5-methylpyrazin-2-yl)methyl)-1H-pyrazole-3-carboxamide (28.0 mg, 0.129 mmol) in MeCN (0.5 mL) under an atmosphere of Ar and the mixture was stirred at RT for 20 min. A solution of 4-(difluoromethoxy)benzene-1-sulfonyl chloride (37.6 mg, 0.155 mmol) in MeCN (0.5 mL) was added and the reaction mixture was stirred at RT for 12 hrs. The crude reaction mixture was concentrated under reduced pressure and the residue was dissolved in DMSO (1.0 mL) and filtered. The crude product was purified by preparative HPLC (26 mg), LC-MS method D: t_{R}=0.61 min, m/z=423.8 [M+H]⁺.

Compound 64, 66-84 and 86 were prepared analogously from the corresponding starting materials.

### Compounds of the Invention

TABLE 1

| | | | | LCMS | | |
|---|---|---|---|---|---|---|
| | | | | Ob- | Reten- | |
| | | | LCMS | served | tion | |
| | Chemical Name | CHEMISTRY | Method | Mass | Time | NMR |
| 1 | 1-(4-methoxy-3- methylbenzene-1- sulfonyl)-N-[(5- methylpyrazin-2- yl)methyl]-1H- pyrazole- | 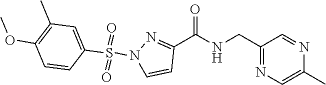 | method A | 402 | 2.11 | ¹H NMR (CDCl₃ 400 MHz): δ 8.52 (s, 1H), 8.42 (s, 1H), 8.11 (d, 1H), 7.88 (dd, 1H), 7.76 (d, 1H), 7.63 (brs, 1H), 6.93-6.91 |
| | 3-carboxamide | | | | | (m, 2H), 4.71 (d, 2H), |
| | | | | | | 3.91 (s, 3H), 2.57 (s, |
| | | | | | | 3H), 2.25 (s, 3H). |
| 2 | 1-(4-methoxy-2- methylbenzene-1- sulfonyl)-N-[(5- methylpyrazin-2- yl)methyl]-1H- | 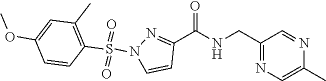 | method A | 402.1 | 2.18 | ¹H NMR (CDCl₃ 400 MHz): δ 8.49 (s, 1H), 8.37 (s, 1H), 8.14 (d, 1H), 8.07 (d, 1H), 7.63 (brt, 1H), 6.90 |
| | pyrazole- | | | | | (d, 1H), 6.85 (dd, 2.8 |
| | 3-carboxamide | | | | | Hz, 1H), 6.77 (d, 1H), |
| | | | | | | 4.67 (d, 2H), 3.85 (s, |
| | | | | | | 3H), 2.54 (6H). |
| 3 | 1-(4-methoxy-2- methylbenzene-1- sulfonyl)-N-[(pyrazin-2- yl)methyl]-1H-pyrazole- 3-carboxamide | 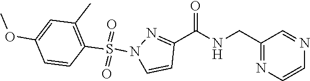 [SMILES: COc1ccc(S(=O)(=O)n2ccc(C(=O)NCc3cnccn3)n2)c(C)c1] | method A | 388.1 | 2.12 | ¹H NMR (CDCl₃ 400 MHz): δ 8.64 (s, 1H), 8.51 (d, 2H), 8.16 (s, 1H), 8.10 (d, 1H), 7.64 (brs, 1H), 6.93 (s, |
| | | | | | | 1H), 6.87 (d, 1H), 6.79 |
| | | | | | | (s, 1H), 4.73 (d, 2H), |
| | | | | | | 3.86 (s, 3H), 2.56 (s, |
| | | | | | | 3H). |
| 4 | 1-(4-methoxy-3- methylbenzene-1- sulfonyl)-N-[(pyrazin-2- yl)methyl]-1H-pyrazole- 3-carboxamide | 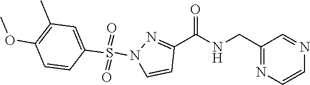 [SMILES: COc1ccc(S(=O)(=O)n2ccc(C(=O)NCc3cnccn3)n2)cc1C] | method A | 388.1 | 2.16 | ¹H NMR (CDCl₃ 400 MHz): δ 8.64 (s, 1H), 8.54-8.50 (m, 2H), 8.12 (d, 1H), 7.88 (dd, 1H), 7.76-7.73 (m, 2H), 6.93-6.91 |
| | | | | | | (m, 2H), 4.75 (d, 2H), |
| | | | | | | 3.90 (s, 3H), 2.24 (s, |
| | | | | | | 3H). |
| 5 | 1-(4-methoxybenzene-1- sulfonyl)-N-[(5- methylpyrazin-2- yl)methyl]-1H-pyrazole- 3-carboxamide | 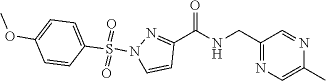 | method B | 388.1 | 1.93 | ¹H NMR (DMSO-d6 400 MHz): δ 9.15 (t, 1H), 8.63 (d, 1H), 8.52 (s, 2H), 8.09-8.05 (m, 2H), 7.30-7.27 (m, |
| | | | | | | 2H), 7.00 (d, 1H), 4.59 |
| | | | | | | (d, 2H), 3.94 (s, 3H), |
| | | | | | | 2.54 (s, 3H). |
| 6 | 1-(4-methoxybenzene-1- sulfonyl)-N-[(4-methyl- 1,3-thiazol-2-yl)methyl]- 1H-pyrazole-3- carboxamide | 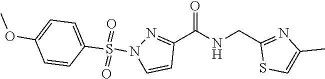 | method A | 393 | 2.08 | 1H NMR (DMSO-d6 400 MHz): δ 9.27 (t, 1H), 8.53 (d, 1H), 7.98-7.95 (m, 2H), 7.19-7.15 (m, 2H), |
| | | | | | | 7.09 (s, 1H), 6.90 (d, |
| | | | | | | 1H), 4.57 (d, 2H), 3.83 |
| | | | | | | (s, 3H), 2.28 (s, 3H). |
| 7 | 1-(4-methoxybenzene-1- sulfonyl)-N-[(pyrimidin- 5-yl)methyl]-1H- pyrazole-3-carboxamide | 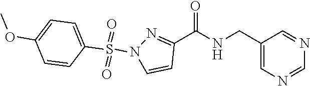 [SMILES: COc1ccc(S(=O)(=O)n2ccc(C(=O)NCc3cncnc3)n2)cc1] | method A | 374 | 1.81 | 1H NMR (DMSO-d6 400 MHz): δ 9.10 (t, 1H), 9.04 (s, 1H), 8.70 (s, 2H), 8.52 (t, 1H), 7.97-7.93 (m, 2H), |
| | | | | | | 7.18-7.15 (m, 2H), |
| | | | | | | 6.87 (d, 1H), 4.39 (d, |
| | | | | | | 2H), 3.83 (s, 3H). |
| 8 | 1-(4-methoxybenzene-1- sulfonyl)-N-[(1,2-oxazol- 3-yl)methyl]-1H- pyrazole-3-carboxamide | 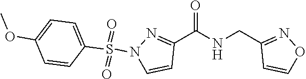 [SMILES: COc1ccc(S(=O)(=O)n2ccc(C(=O)NCc3ccon3)n2)cc1] | method B | 363.1 | 1.99 | ¹H NMR (DMSO-d6 400 MHz): δ 9.04 (t, 1H), 8.78 (d, 1H), 8.52 (d, 1H), 7.98-7.94 (m, 2H), 7.19-7.15 (m, |
| | | | | | | 2H), 6.89 (d, 1H), 6.44 |
| | | | | | | (d, 1H), 4.44 (d, 2H), |
| | | | | | | 3.83 (s, 3H). |
| 9 | 1-(4-methoxybenzene-1- sulfonyl)-N-[(1,2-oxazol- 4-yl)methyl]-1H- pyrazole-3-carboxamide | 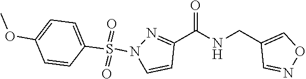 [SMILES: COc1ccc(S(=O)(=O)n2ccc(C(=O)NCc3cnoc3)n2)cc1] | method B | 363.1 | 1.98 | 1H NMR (CDCl₃ 400 MHz): δ 8.43 (s, 1H), 8.31 (s, 1H), 8.10 (1H), 7.92 (d, 2H), 7.25 (brs, 1H), 6.99 |
| | | | | | | (d, 2H), 6.90 (d, 1H), |
| | | | | | | 4.44 (d, 2H), 3.87 (s, |
| | | | | | | 3H). |
| 10 | 1-(4-methoxybenzene-1- sulfonyl)-N-[(1,2-oxazol- 5-yl)methyl]-1H- pyrazole-3-carboxamide | 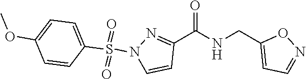 [SMILES: COc1ccc(S(=O)(=O)n2ccc(C(=O)NCc3ccno3)n2)cc1] | method A | 363 | 1.99 | 1H NMR (CDCl₃ 400 MHz): δ 8.19 (s, 1H), 8.12 (s, 1H), 7.95 (d, 2H), 7.36 (brs, 1H), 7.01 (d, 2H), 6.90 |
| | | | | | | (d, 1H), 6.24 (s, 1H), |
| | | | | | | 4.72 (d, 2H), 3.88 (s, |
| | | | | | | 3H). |
| 11 | 1-(4-methoxybenzene-1- sulfonyl)-N-[(2- methylpyrimidin-5- yl)methyl]-1H-pyrazole- 3-carboxamide | 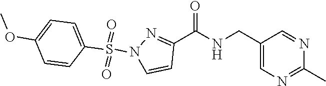 | method A | 388 | 1.83 | ¹H NMR (CDCl₃ 400 MHz): δ 8.56 (s, 2H), 8.04 (s, 1H), 7.84 (d, 2H), 7.35 (s, 1H), 6.92 (d, 2H), 6.84 (s, 1H), 4.47 (d, 2H), 3.80 |
| | | | | | | (s, 3H), 2.65 (s, 3H). |
| 12 | 1-(4-methoxybenzene-1- sulfonyl)-N-[(1,3-oxazol- 4-yl)methyl]-1H- pyrazole-3-carboxamide | 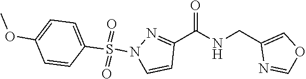 [SMILES: COc1ccc(S(=O)(=O)n2ccc(C(=O)NCc3cocn3)n2)cc1] | method A | 363 | 1.91 | ¹H NMR (CDCl₃ 400 MHz): δ 8.06 (d, 1H), 7.89 (d, 2H), 7.84 (s, 1H), 7.62 (s, 1H), 7.42 (brs, 1H), 6.95 |
| | | | | | | (d, 2H), 6.85 (d, 1H), |
| | | | | | | 4.47 (d, 2H), 3.83 (s, |
| | | | | | | 3H). |
| 13 | 1-(4-methoxybenzene-1- sulfonyl)-N-[(1-methyl- 1H-1,2,4-triazol-3- yl)methyl]-1H-pyrazole- 3-carboxamide | 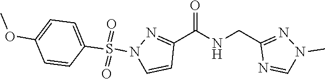 | method A | 377 | 1.74 | ¹H NMR (CDCl₃ 400 MHz): δ 8.08 (s, 1H), 7.98 (s, 1H), 7.94 (d, 2H), 7.45 (s, 1H), 6.99 (d, 2H), 6.90 (s, |
| | | | | | | 1H), 4.67 (d, 2H), 3.89 |
| | | | | | | (s, 3H), 3.87 (s, 3H). |
| 14 | 1-(benzenesulfonyl)-N- [(3-methyl-1,2-oxazol-5- yl)methyl]-1H-pyrazole- 3-carboxamide | 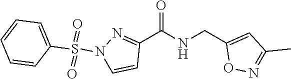 [SMILES: Cc1cc(CNC(=O)c2ccn(S(=O)(=O)c3ccccc3)n2)on1] | method B | 347.1 | 1.99 | ¹H NMR (DMSO-d6 400 MHz): δ 9.11(t, 1H), 8.58 (d, 1H), 8.02 (d, 2H), 7.80 (t, 1H), 7.66 (t, 2H), 6.92 (d, |
| | | | | | | 1H), 6.13 (s, 1H), 4.43 |
| | | | | | | (d, 2H), 2.13 (s, 3H). |
| 15 | 1-(4-methylbenzene-1- sulfonyl)-N-[(5- methylpyrimidin-2- yl)methyl]-1H-pyrazole- 3-carboxamide | 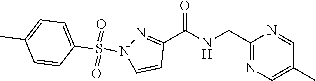 | method B | 372 | 1.86 | ¹H NMR (DMSO-d6 400 MHz): δ 8.86 (t, 1H), 8.57 (m, 3H), 7.93 (d, 2H), 7.49 (d, 2H), 6.91 (d, 1H), 4.53 |
| | | | | | | (d, 2H), 2.39 (s, 3H), |
| | | | | | | 2.22 (s, 3H). |
| 16 | 1-(4-methylbenzene-1- sulfonyl)-N-[(5- methylpyridin-2- yl)methyl]-1H-pyrazole- 3-carboxamide | 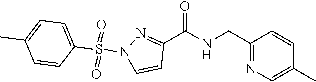 | method A | 371 | 1.71 | ¹H NMR (DMSO-d6 400 MHz): δ 8.98 (t, 1H), 8.56 (d, 1H), 8.31 (s, 1H), 7.93 (d, 2H), 7.53 (dd, 1H), 7.48 (d, |
| | | | | | | 2H), 7.15 (d, 1H), 6.92 |
| | | | | | | (d, 1H), 4.44 (d, 2H), |
| | | | | | | 2.38 (s, 3H), 2.23 (s, |
| | | | | | | 3H). |
| 17 | 1-(2-methylbenzene-1- sulfonyl)-N-[(5-methyl- 1,3-oxazol-2-yl)methyl]- 1H-pyrazole-3- carboxamide | 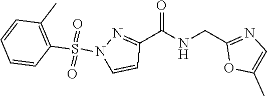 [SMILES: Cc1cnc(CNC(=O)c2ccn(S(=O)(=O)c3ccccc3C)n2)o1] | method B | 361 | 1.84 | ¹H NMR (CDCl₃ 400 MHz): δ 8.18 (d, 1H), 8.11 (d, 1H), 7.57-7.55 (m, 1H), 7.41 (t, 1H), 7.34-7.29 (m, 2H), 6.95 (d, 1H), |
| | | | | | | 6.64 (s, 1H), 4.63 (d, |
| | | | | | | 2H), 2.58 (s, 3H), 2.28 |
| | | | | | | (s, 3H). |
| 18 | 1-(3-methylbenzene-1- sulfonyl)-N-[(5-methyl- 1,3-oxazol-2-yl)methyl]- 1H-pyrazole-3- carboxamide | 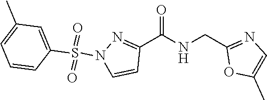 [SMILES: Cc1cccc(S(=O)(=O)n2ccc(C(=O)NCc3ncc(C)o3)n2)c1] | method B | 361.1 | 2.17 | ¹H NMR (CDCl₃ 400 MHz): δ 8.13 (d, 1H), 7.81 (m, 2H), 7.49-7.45 (m, 2H), 7.38 (s, 1H), 6.94 (d, 1H), 6.67 (s, 1H), 4.65 (d, 2H), 2.45 (s, 3H), |
| | | | | | | 2.30 (s, 3H). |
| 19 | 1-(3-methylbenzene-1- sulfonyl)-N-[(3-methyl- 1,2-oxazol-5-yl)methyl]- 1H-pyrazole-3- carboxamide | 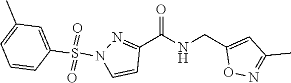 [SMILES: Cc1cccc(S(=O)(=O)n2ccc(C(=O)NCc3cc(C)no3)n2)c1] | method B | 361.1 | 2.22 | ¹H NMR (CDCl₃ 400 MHz): δ 8.13 (d, 1H), 7.81-7.80 (m, 2H), 7.48-7.45 (m, 2H), 7.36-7.34 (m, |
| | | | | | | 1H), 6.92 (d, 1H), 6.06 |
| | | | | | | (s, 1H), 4.64 (d, 2H), |
| | | | | | | 2.44 (s, 3H), 2.26 (s, |
| | | | | | | 3H). |
| 20 | 1-(3-methylbenzene-1- sulfonyl)-N-[(5- methylpyridin-2- yl)methyl]-1H-pyrazole- 3-carboxamide | 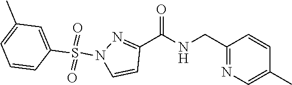 [SMILES: Cc1ccc(CNC(=O)c2ccn(S(=O)(=O)c3cccc(C)c3)n2)nc1] | method B | 371.1 | 2.29 | ¹H NMR (CDCl₃ 400 MHz): δ 8.39 (s, 1H), 8.11 (d, 1H), 7.84-7.80 (m, 3H), 7.55 (d, 1H), 7.47- 7.45 (m, 2H), 7.29 (d, |
| | | | | | | 1H), 6.91 (d, 1H), 4.69 |
| | | | | | | (d, 2H), 2.44 (s, 3H), |
| | | | | | | 2.35 (s, 3H). |
| 21 | 1-(3-methylbenzene-1- sulfonyl)-N-[(1-methyl- 1H-pyrazol-4-yl)methyl]- 1H-pyrazole-3- carboxamide | 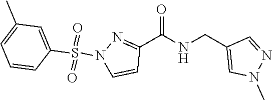 [SMILES: Cc1cccc(S(=O)(=O)n2ccc(C(=O)NCc3cnn(C)c3)n2)c1] | method B | 360.1 | 2.02 | ¹H NMR (CDCl₃ 400 MHz): δ 8.11 (d, 1H), 7.77 (m, 2H), 7.47-7.44 (m, 3H), 7.38 (s, 1H), 7.08 (brs, 1H), 6.92 (d, 1H), 4.42 (d, 2H), |
| | | | | | | 3.87 (s, 3H), 2.43 |
| | | | | | | (s, 3H). |
| 22 | 1-(3-methylbenzene-1- sulfonyl)-N-[(1-methyl- 1H-pyrazol-3-yl)methyl]- 1H-pyrazole-3- carboxamide | 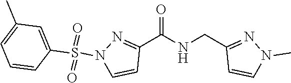 [SMILES: Cc1cccc(S(=O)(=O)n2ccc(C(=O)NCc3ccn(C)n3)n2)c1] | method B | 360.1 | 2.06 | ¹H NMR (CDCl₃, 400 MHz): δ 8.11 (d, 1H), 7.79-7.78 (m, 2H), 7.47-7.44 (m, 2H), 7.29 (m, 1H), 6.92 (d, 1H), 6.20 (d, |
| | | | | | | 1H), 4.57 (d, 2H), 3.81 |
| | | | | | | (s, 3H), 2.44 (s, 3H). |
| 23 | 1-(2-methylbenzene-1- sulfonyl)-N-[(3-methyl- 1,2,4-oxadiazol-5- yl)methyl]-1H-pyrazole- 3-carboxamide | 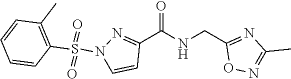 [SMILES: Cc1noc(CNC(=O)c2ccn(S(=O)(=O)c3ccccc3C)n2)n1] | method B | 362.1 | 2.13 | ¹H NMR (CDCl₃ 400 MHz): δ 8.20 (d, 1H), 8.12 (d, 1H), 7.58-7.56 (m, 1H), 7.44-7.40 (m, 2H), |
| | | | | | | 7.33 (d, 1H), 6.95 (d, |
| | | | | | | 1H), 4.78 (d, 2H), 2.59 |
| | | | | | | (s, 3H), 2.37 (s, 3H). |
| 24 | 1-(4-methylbenzene-1- sulfonyl)-N-[(pyrazin-2- yl)methyl]-1H-pyrazole- 3-carboxamide | 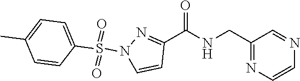 [SMILES: Cc1ccc(S(=O)(=O)n2ccc(C(=O)NCc3cnccn3)n2)cc1] | method B | 358.1 | 2 | ¹H NMR (DMSO-d6 400 MHz): δ 9.11 (t, 1H), 8.58-8.54 (m, 3H), 8.50 (s, 1H), 7.92 (d, 2H), 7.48 (d, 2H), |
| | | | | | | 6.92 (d, 1H), 4.54 (d, |
| | | | | | | 2H), 2.38 (s, 3H). |
| 25 | 1-(2-methylbenzene-1- sulfonyl)-N-[(5- methylpyrimidin-2- yl)methyl]-1H-pyrazole- 3-carboxamide | 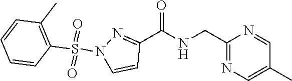 [SMILES: Cc1cnc(CNC(=O)c2ccn(S(=O)(=O)c3ccccc3C)n2)nc1] | method B | 372.1 | 2.11 | ¹H NMR (CDCl₃ 400 MHz): δ 8.52 (s, 2H), 8.16 (d, 1H), 8.10 (d, 1H), 7.91 (brs, 1H), 7.57-7.53 (m, |
| | | | | | | 1H), 7.39 (t, 1H), 7.32 |
| | | | | | | (d, 1H), 6.94 (d, 1H), |
| | | | | | | 4.77 (d, 2H), 2.66 (s, |
| | | | | | | 3H), 2.30 (s, 3H). |
| 26 | 1-(2-methylbenzene-1- sulfonyl)-N-[(1-methyl- 1H-pyrazol-3-yl)methyl]- 1H-pyrazole-3- carboxamide | 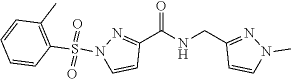 [SMILES: Cc1ccccc1S(=O)(=O)n1ccc(C(=O)NCc2ccn(C)n2)n1] | method B | 360 | 1.81 | ¹H NMR (CDCl₃ 400 MHz): δ 8.16 (d, 1H), 8.07 (d, 1H), 7.54 (t, 1H), 7.39 (t, 1H), 7.31 (d, 1H), 7.27 (d, |
| | | | | | | 1H), 7.20 (s, 1H), 6.93 |
| | | | | | | (d, 1H), 6.17 (d, 1H), |
| | | | | | | 4.54 (d, 2H), 3.85 (s, |
| | | | | | | 3H), 2.57 (s, 3H). |
| 27 | 1-(2-methylbenzene-1- sulfonyl)-N-[(5- methylpyridin-2- yl)methyl]-1H-pyrazole- 3-carboxamide | 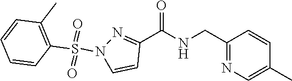 [SMILES: Cc1ccc(CNC(=O)c2ccn(S(=O)(=O)c3ccccc3C)n2)nc1] | method B | 371 | 2.02 | ¹H NMR (CDCl₃ 400 MHz): δ 8.36 (s, 1H), 8.18 (d, 1H), 8.11 (d, 1H), 7.75 (m, 1H), 7.58-7.54 (m, 1H), |
| | | | | | | 7.46-7.41 (m, 2H), |
| | | | | | | 7.32 (d, 1H), 7.19 (d, |
| | | | | | | 1H), 6.94 (d, 1H), 4.64 |
| | | | | | | (d, 2H), 2.63 (s, 3H), |
| | | | | | | 2.32 (s, 3H). |
| 28 | 1-(2-methylbenzene-1- sulfonyl)-N-[(1-methyl- 1H-pyrazol-4-yl)methyl]- 1H-pyrazole-3- carboxamide | 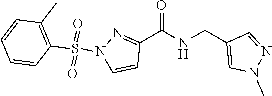 [SMILES: Cc1ccccc1S(=O)(=O)n1ccc(C(=O)NCc2cnn(C)c2)n1] | method B | 360 | 1.77 | ¹H NMR (CDCl₃ 400 MHz): δ 8.18 (d, 1H), 8.08 (d, 1H), 7.57 (t, 1H), 7.42-7.31 (m, 4H), 7.02 (s, 1H), 6.94 (d, 1H), 4.40 (d, 2H), |
| | | | | | | 3.86 (s, 3H), 2.56 (s, |
| | | | | | | 3H). |
| 29 | 1-(2-methylbenzene-1- sulfonyl)-N-[(3-methyl- 1,2-oxazol-5-yl)methyl]- 1H-pyrazole-3- carboxamide | 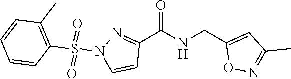 [SMILES: Cc1cc(CNC(=O)c2ccn(S(=O)(=O)c3ccccc3C)n2)on1] | method B | 361 | 1.9 | ¹H NMR (DMSO-d6 400 MHz): δ 9.06 (t, 1H), 8.67 (d, 1H), 8.05 (d, 1H), 7.72-7.70 (m, 1H), 7.57-7.49 (m, |
| | | | | | | 2H), 6.97 (d, 1H), 6.15 |
| | | | | | | (s, 1H), 4.45 (d, 2H), |
| | | | | | | 2.51 (s, 3H), 2.16 (s, |
| | | | | | | 3H). |
| 30 | 1-(4-methylbenzene-1- sulfonyl)-N-[(5-methyl- 1,3-oxazol-2-yl)methyl]- 1H-pyrazole-3- carboxamide | 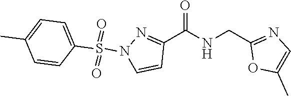 [SMILES: Cc1ccc(S(=O)(=O)n2ccc(C(=O)NCc3ncc(C)o3)n2)cc1] | method B | 361 | 1.91 | ¹H NMR (DMSO-d6 400 MHz): δ 9.05 (m, 1H), 8.56 (d, 1H), 7.92 (d, 2H), 7.48 (d, 2H), 6.91 (d, 1H), 6.71 (s, 1H), 4.41 (d, 2H), 2.38 |
| | | | | | | (s, 3H), 2.22 (s, 3H). |
| 31 | 1-(4-fluorobenzene-1- sulfonyl)-N-[(3-methyl- 1,2-oxazol-5-yl)methyl]- 1H-pyrazole-3- carboxamide | 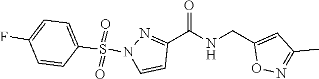 [SMILES: Cc1cc(CNC(=O)c2ccn(S(=O)(=O)c3ccc(F)cc3)n2)on1] | Method B | 365 | 1.85 | ¹H NMR (DMSO-d⁶ 400 MHz): δ 9.16 (t, 1H), 8.62 (d, 1H), 8.18-8.13 (m, 2H), 7.57 (t, 2H), 6.97 (d, |
| | | | | | | 1H), 6.18 (s, 1H), 4.48 |
| | | | | | | (d, 2H), 2.18 (s, 3H). |
| 32 | 4-methyl-1-(4- methylbenzene-1- sulfonyl)-N-[(5- methylpyrazin-2- yl)methyl]-1H-pyrazole- 3-carboxamide | 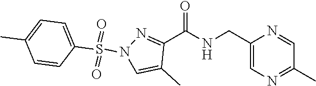 | method D | 386.1 | 0.66 | ¹H NMR (CDCl₃, 600 MHz) δ 8.49 (d, 1H), 8.39 (d, 1H), 7.85 (dd, 2H), 7.84 (s, 1H), 7.63 (t, 1H), 7.34-7.31 (m, 2H), 4.65 (d, 2H), |
| | | | | | | 2.54 (s, 3H), 2.42 (s, |
| | | | | | | 3H), 2.29 (d, 3H). |
| 33 | 1-(4-methylbenzene-1- sulfonyl)-N-[(1,2-thiazol- 4-yl)methyl]-1H- pyrazole-3-carboxamide | 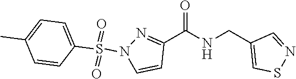 [SMILES: Cc1ccc(S(=O)(=O)n2ccc(C(=O)NCc3cnsc3)n2)cc1] | method B | 363.1 | 2.15 | ¹H NMR (DMSO-d6 400 MHz): δ 9.08- 9.06 (m, 1H), 8.80 (s, 1H), 8.57 (d, 1H), 8.49 (s, 1H), 7.92 (d, 2H), |
| | | | | | | 7.50 (d, 2H), 6.92 (d, |
| | | | | | | 1H), 4.47 (d, 2H), 2.40 |
| | | | | | | (s, 3H). |
| 34 | 1-(4-fluorobenzene-1- sulfonyl)-N-[(5- methylpyridin-2- yl)methyl]-1H-pyrazole- 3-carboxamide | 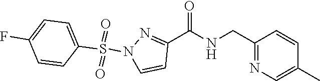 [SMILES: Cc1ccc(CNC(=O)c2ccn(S(=O)(=O)c3ccc(F)cc3)n2)nc1] | method B | 375.1 | 2.16 | ¹H NMR (DMSO-d6 400 MHz): δ 9.03 (t, 1H), 8.62 (d, 1H), 8.33 (s, 1H), 8.19-8.15 (m, 2H), 7.59-7.54 (m, |
| | | | | | | 3H), 7.18 (d, 1H), 6.97 |
| | | | | | | (d, 1H), 4.47 (d, 2H), |
| | | | | | | 2.26 (s, 3H). |
| 35 | 1-(4-fluorobenzene-1- sulfonyl)-N-[(1-methyl- 1H-pyrazol-3-yl)methyl]- 1H-pyrazole-3- carboxamide | 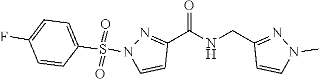 [SMILES: Cn1ccc(CNC(=O)c2ccn(S(=O)(=O)c3ccc(F)cc3)n2)n1] | method B | 364.1 | 1.93 | ¹H NMR (DMSO-d6 400 MHz): δ 8.84 (t, 1H), 8.59 (d, 1H), 8.17-8.14 (m, 2H), 7.58-7.54 (m, 3H), |
| | | | | | | 6.95 (d, 1H), 6.85 (d, |
| | | | | | | 1H), 4.33 (d, 2H), 3.77 |
| | | | | | | (s, 3H). |
| 36 | 1-(4-methoxybenzene-1- sulfonyl)-N-[(5-methyl- 1,3-oxazol-2-yl)methyl]- 1H-pyrazole-3- carboxamide | 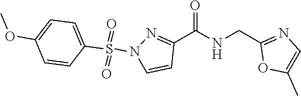 [SMILES: COc1ccc(S(=O)(=O)n2ccc(C(=O)NCc3ncc(C)o3)n2)cc1] | method B | 377 | 1.77 | ¹H NMR (CDCl₃ 400 MHz): δ 8.10 (d, 1H), 7.96 (d, 2H), 7.38 (m, 1H), 7.01 (d, 2H), 6.91 (d, 1H), 6.67 (s, 1H), 4.65 (d, 2H), 3.88 |
| | | | | | | (s, 3H), 2.30 (s, 3H). |
| 37 | 1-(4-methoxybenzene-1- sulfonyl)-N-[(5- methylpyrimidin-2- yl)methyl]-1H-pyrazole- 3-carboxamide | 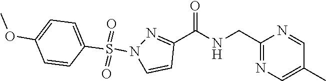 | method B | 388 | 1.72 | ¹H NMR (CDCl₃ 400 MHz): δ 8.57 (s, 2H), 8.10 (d, 1H), 7.98 (dd, 2H), 7.91 (m, 1H), 7.00 (d, 2H), 6.91 (d, 1H), 4.81 (d, 2H), |
| | | | | | | 3.88 (s, 3H), 2.33 (s, |
| | | | | | | 3H). |
| 38 | 1-(4-methoxybenzene-1- sulfonyl)-N-[(3-methyl- 1,2-oxazol-5-yl)methyl]- 1H-pyrazole-3- carboxamide | 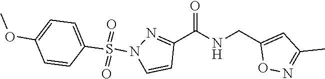 | method B | 377 | 1.8 | ¹H NMR (CDCl₃ 400 MHz): δ 8.11 (d, 1H), 7.96 (dd, 2H), 7.02 (d, 2H), 6.90 (d, 1H), 6.07 (s, 1H), 4.65 |
| | | | | | | (d, 2H), 3.89 (s, 3H), |
| | | | | | | 2.27 (s, 3H). |
| 39 | 1-(4-methylbenzene-1- sulfonyl)-N-[(1-methyl- 1H-pyrazol-3-yl)methyl]- 1H-pyrazole-3- carboxamide | 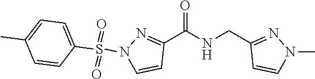 | method B | 360.1 | 2.07 | ¹H NMR (DMSO-d6 400 MHz): δ 8.78- 8.76 (m, 1H), 8.55 (d, 1H), 7.93 (d, 2H), 7.55 (d, 1H), 7.49 (d, 2H), |
| | | | | | | 6.92 (d, 1H), 6.08 (d, |
| | | | | | | 1H), 4.32 (d, 2H), 3.76 |
| | | | | | | (s, 3H), 2.40 (s, 3H). |
| 40 | 1-(4-methoxybenzene-1- sulfonyl)-N-[(5- methylpyridin-2- yl)methyl]-1H-pyrazole- 3-carboxamide | 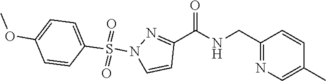 | method B | 387 | 1.97 | ¹H NMR (CDCl₃ 400 MHz): δ 8.39 (s, 1H), 8.09 (d, 1H), 7.96 (d, 2H), 7.75 (br s, 1H), 7.47 (d, 1H), 7.22 |
| | | | | | | (d, 1H), 7.00 (d, 2H), |
| | | | | | | 6.89 (d, 1H), 4.65 (d, |
| | | | | | | 2H), 3.87 (s, 3H), 2.33 |
| | | | | | | (s, 3H). |
| 41 | 1-(4-methoxybenzene-1- sulfonyl)-N-[(1-methyl- 1H-pyrazol-4-yl)methyl]- 1H-pyrazole-3- carboxamide | 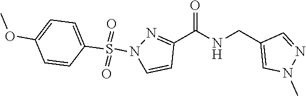 [SMILES: COc1ccc(S(=O)(=O)n2ccc(C(=O)NCc3cnn(C)c3)n2)cc1] | method B | 376 | 1.72 | ¹H NMR (CDCl₃ 400 MHz): δ 8.08 (d, 1H), 7.92 (d, 2H), 7.45 (s, 1H), 7.39 (s, 1H), 7.08 (m, 1H), 6.99 (d, 2H), 6.89 (d, 1H), 4.41 |
| | | | | | | (d, 2H), 3.87 (s, 6H). |
| 42 | 1-(4-methoxybenzene-1- sulfonyl)-N-[(3-methyl- 1,2,4-oxadiazol-5- yl)methyl]-1H-pyrazole- 3-carboxamide | 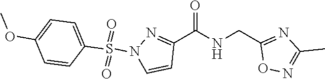 | method B | 378 | 1.8 | ¹H NMR (CDCl₃ 400 MHz): δ 8.12 (d, 1H), 7.96 (d, 2H), 7.46 (m, 1H), 7.02 (d, 2H), 6.91 (d, 1H), 4.80 (d, |
| | | | | | | 2H), 3.89 (s, 3H), 2.39 |
| | | | | | | (s, 3H). |
| 43 | 1-(4-methoxybenzene-1- sulfonyl)-N-[(pyrazin-2- yl)methyl]-1H-pyrazole- 3-carboxamide | 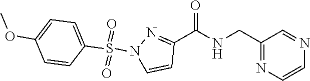 [SMILES: COc1ccc(S(=O)(=O)n2ccc(C(=O)NCc3cnccn3)n2)cc1] | method B | 374 | 1.67 | ¹H NMR (CDCl₃ 400 MHz): δ 8.64 (s, 1H), 8.54 (s, 1H), 8.51 (d, 1H), 8.11 (d, 1H), 7.96 (d, 2H), 7.68 (m, 1H), 7.00 (d, 2H), 6.91 |
| | | | | | | (d, 1H), 4.75 (d, 2H), |
| | | | | | | 3.88 (s, 3H). |
| 44 | 1-(4-methoxybenzene-1- sulfonyl)-N-[(1-methyl- 1H-pyrazol-3-yl)methyl]- 1H-pyrazole-3- carboxamide | 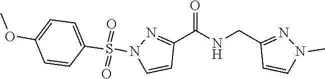 | method B | 376 | 1.73 | ¹H NMR (CDCl₃ 400 MHz): δ 8.07 (d, 1H), 7.95-7.92 (m, 2H), 7.29-7.28 (m, 2H), 7.01-6.98 (m, |
| | | | | | | 2H), 6.88 (d, 1H), 6.20 |
| | | | | | | (m, 1H), 4.57 (d, 2H), |
| | | | | | | 3.88 (s, 3H), 3.87 (s, |
| | | | | | | 3H). |
| 45 | 1-(4-methylbenzene-1- sulfonyl)-N-[(1,3-oxazol- 2-yl)methyl]-1H- pyrazole-3-carboxamide | 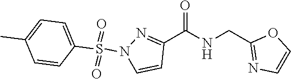 [SMILES: Cc1ccc(S(=O)(=O)n2ccc(C(=O)NCc3ncco3)n2)cc1] | method B | 347 | 1.78 | ¹H NMR (DMSO-d6 400 MHz): δ 9.13- 9.11 (m, 1H), 8.59 (d, 1H), 8.02 (s, 1H), 7.94 (d, 2H), 7.51 (d, 2H), |
| | | | | | | 7.13 (d, 1H), 6.94 (d, |
| | | | | | | 1H), 4.50 (d, 2H), 2.41 |
| | | | | | | (s, 3H). |
| 46 | 1-(4-methylbenzene-1- sulfonyl)-N-[(1-methyl- 1H-1,2,3-triazol-4- yl)methyl]-1H-pyrazole- 3-carboxamide | 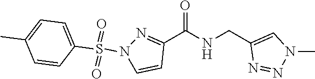 | method B | 361 | 1.65 | ¹H NMR (DMSO-d6 400 MHz): δ 9.03- 8.99 (m, 1H), 8.56 (d, 1H), 7.93 (d, 2H), 7.86 (s, 1H), 7.50 (d, 2H), |
| | | | | | | 6.91 (d, 1H), 4.41 (d, |
| | | | | | | 2H), 3.98 (s, 3H), 2.40 |
| | | | | | | (s, 3H). |
| 47 | 1-(4-methylbenzene-1- sulfonyl)-N-[(1,3,4- thiadiazol-2-yl)methyl]- 1H-pyrazole-3- carboxamide | 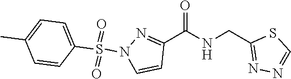 [SMILES: Cc1ccc(S(=O)(=O)n2ccc(C(=O)NCc3nncs3)n2)cc1] | Method B | 364 | 1.97 | ¹H NMR (DMSO-d6 400 MHz): δ 9.51 (s, 1H), 9.44 (t, 1H), 8.60 (d, 1H), 7.94 (d, 2H), 7.50 (d, 2H), 6.95 (d, |
| | | | | | | 1H), 4.81 (d, 2H), 2.40 |
| | | | | | | (s, 3H). |
| 48 | 1-(4-methylbenzene-1- sulfonyl)-N-[(3-methyl- 1,2,4-oxadiazol-5- yl)methyl]-1H-pyrazole- 3-carboxamide | 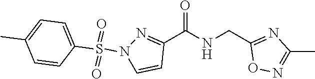 | method B | 362.1 | 2.1 | ¹H NMR (DMSO-d6 400 MHz): δ 9.24- 9.22 (m, 1H), 8.62 (d, 1H), 7.95 (d, 2H), 7.52 (d, 2H), 6.94 (d, 1H), |
| | | | | | | 4.63 (d, 2H), 2.41 (s, |
| | | | | | | 3H), 2.30 (s, 3H). |
| 49 | 1-(4-methylbenzene-1- sulfonyl)-N-[(1,3- oxazol-4-yl)methyl]- 1H-pyrazole- 3-carboxamide | 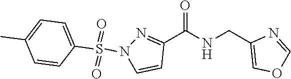 [SMILES: Cc1ccc(S(=O)(=O)n2ccc(C(=O)NCc3cocn3)n2)cc1] | method B | 347.1 | 1.99 | ¹H NMR (DMSO-d6 400 MHz): δ 8.88- 8.86 (m, 1H), 8.56 (d, 1H), 8.29 (s, 1H), 7.94-7.92 (m, 3H), |
| | | | | | | 7.50 (d, 2H), 6.92 (d, |
| | | | | | | 1H), 4.30 (d, 2H), 2.40 |
| | | | | | | (s, 3H). |
| 50 | 1-(4-methylbenzene-1- sulfonyl)-N-[(2- methylpyrimidin-5- yl)methyl]-1H-pyrazole- 3-carboxamide | 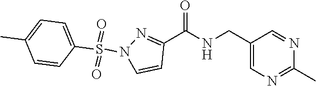 | method B | 372.1 | 1.91 | ¹H NMR (DMSO-d6 400 MHz): δ 9.14- 9.12 (m, 1H), 8.61 (s, 2H), 8.57 (d, 1H) 7.92 (d, 2H), 7.49 (d, 2H), 6.91 (d, 1H), 4.37 (d, |
| | | | | | | 2H), 2.57 (s, 3H), 2.39 |
| | | | | | | (s, 3H). |
| 51 | 1-(4-methylbenzene-1- sulfonyl)-N-[(1,2-oxazol- 4-yl)methyl]-1H- pyrazole-3-carboxamide | 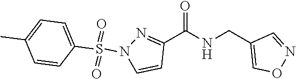 [SMILES: Cc1ccc(S(=O)(=O)n2ccc(C(=O)NCc3cnoc3)n2)cc1] | method B | 347.1 | 2.09 | ¹H NMR (DMSO-d6 400 MHz): δ 8.96- 8.94 (m, 1H), 8.82 (d, 1H), 8.58 (d, 1H), 8.52 (s, 1H), 7.92 (d, 2H), |
| | | | | | | 7.49 (d, 2H), 6.92 (d, |
| | | | | | | 1H), 4.27 (d, 2H), 2.38 |
| | | | | | | (s, 3H). |
| 52 | 1-(4-methylbenzene-1- sulfonyl)-N-[(1,3-thiazol- 4-yl)methyl]-1H- pyrazole-3-carboxamide | 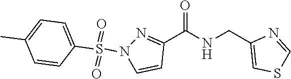 [SMILES: Cc1ccc(S(=O)(=O)n2ccc(C(=O)NCc3cscn3)n2)cc1] | method B | 363.1 | 2.07 | ¹H NMR (DMSO-d6 400 MHz): δ 9.03- 9.00 (m, 2H), 8.57 (d, 1H), 7.94 (d, 2H), 7.49 (d, 2H), 7.42 (s, 1H), |
| | | | | | | 6.95 (d, 1H), 4.55 (d, |
| | | | | | | 2H), 2.38 (s, 3H). |
| 53 | 1-(4-methylbenzene-1- sulfonyl)-N-[(1-methyl- 1H-imidazol-4- yl)methyl]-1H-pyrazole- 3-carboxamide | 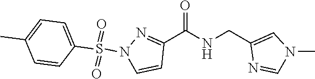 | method A | 360.1 | 1.7 | ¹H NMR (DMSO-d6 400 MHz): δ 8.63- 8.62 (m, 1H), 8.54 (d, 1H), 7.93 (d, 2H), 7.50-7.46 (m, 3H), |
| | | | | | | 6.92 (d, 2H), 4.25 (d, |
| | | | | | | 2H), 3.57 (s, 3H), 2.39 |
| | | | | | | (s, 3H). |
| 54 | 1-(4-methylbenzene-1- sulfonyl)-N-[(pyrimidin- 5-yl)methyl]-1H- pyrazole-3-carboxamide | 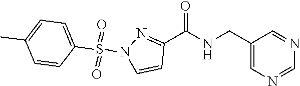 [SMILES: Cc1ccc(S(=O)(=O)n2ccc(C(=O)NCc3cncnc3)n2)cc1] | method B | 358.2 | 1.88 | ¹H NMR (DMSO-d6 400 MHz): δ 9.13 (t, 1H), 9.05 (s, 1H), 8.72-8.71 (m, 2H), 8.56 (d, 1H), 7.90 (d, 2H), 7.48 (d, 2H), |
| | | | | | | 6.90 (d, 1H), 4.40 |
| | | | | | | (d, 2H), 2.38 (s, 3H). |
| 55 | 1-(4-methylbenzene-1- sulfonyl)-N-[(1,2-oxazol- 5-yl)methyl]-1H- pyrazole-3-carboxamide | 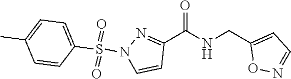 [SMILES: Cc1ccc(S(=O)(=O)n2ccc(C(=O)NCc3ccno3)n2)cc1] | method B | 346.9 | 1.86 | ¹H NMR (DMSO-d6 400 MHz): δ 9.16 (s, 1H), 8.58 (s, 1H), 8.46 (s, 1H), 7.94 (d, 2H), 7.50 (d, 2H), 6.94 (s, |
| | | | | | | 1H), 6.32 (s, 1H), 4.55 |
| | | | | | | (d, 2H), 2.39 (s, 3H). |
| 56 | 1-(4-methylbenzene-1- sulfonyl)-N-[(3-methyl- 1,2-oxazol-5-yl)methyl]- 1H-pyrazole-3- carboxamide | 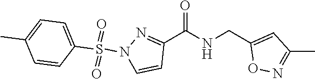 | method B | 360.9 | 1.91 | ¹H NMR (DMSO-d6 400 MHz): δ 9.12 (s, 1H), 8.58 (s, 1H), 7.94 (d, 2H), 7.50 (d, 2H), 6.94 (s, 1H), 6.16 (s, |
| | | | | | | 1H), 4.47 (d, 2H), 2.40 |
| | | | | | | (s, 3H), 2.17 (s, 3H). |
| 57 | 1-(4-methylbenzene-1- sulfonyl)-N-[(1,2,4- oxadiazol-3-yl)methyl]- 1H-pyrazole-3- carboxamide | 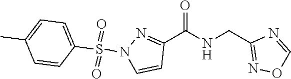 [SMILES: Cc1ccc(S(=O)(=O)n2ccc(C(=O)NCc3ncon3)n2)cc1] | method B | 348.1 | 1.99 | ¹H NMR (DMSO-d6 400 MHz): δ 9.53 (s, 1H), 9.15-9.13 (m, 1H), 8.59 (d, 1H), 7.94 (d, 2H), 7.50 (d, 2H), |
| | | | | | | 6.94 (d, 1H), 4.56 (d, |
| | | | | | | 2H), 2.40 (s, 3H). |
| 58 | 1-(4-methylbenzene-1- sulfonyl)-N- [(1,2-oxazol- 3-yl)methyl]-1H- pyrazole-3-carboxamide | 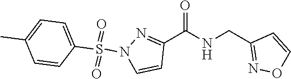 [SMILES: Cc1ccc(S(=O)(=O)n2ccc(C(=O)NCc3ccon3)n2)cc1] | method B | 347.1 | 2.1 | ¹H NMR (DMSO-d6 400 MHz): δ 9.09 (brs, 1H), 8.81 (s, 1H), 8.58 (s, 1H), 7.94 (d, 2H), 7.50 (d, 2H), 6.94 |
| | | | | | | (s, 1H), 6.47 (s, 1H), |
| | | | | | | 4.47 (d, , 2H), 2.40 (s, |
| | | | | | | 3H). |
| 59 | 1-(4-methylbenzene-1- sulfonyl)-N- [(1,3-thiazol- 2-yl)methyl]-1H- pyrazole-3-carboxamide | 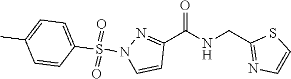 [SMILES: Cc1ccc(S(=O)(=O)n2ccc(C(=O)NCc3nccs3)n2)cc1] | method B | 363.1 | 2.15 | ¹H NMR (DMSO-d6 400 MHz): δ 9.36- 9.34 (m, 1H), 8.59 (d, 1H), 7.95 (d, 2H), 7.71 (d, 1H), 7.61 (d, 1H), |
| | | | | | | 7.51 (d, 2H), 6.96 (d, |
| | | | | | | 1H), 4.68 (d, 2H), 2.40 |
| | | | | | | (s, 3H). |
| 60 | 1-(4-methylbenzene-1- sulfonyl)-N-[(1-methyl- 1H-1,2,4-triazol-3- yl)methyl]-1H-pyrazole- 3-carboxamide | 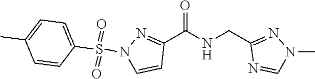 | method B | 361.1 | 1.83 | ¹H NMR (DMSO-d6 400 MHz): δ 8.83 (t, 1H), 8.56 (d, 1H), 8.34 (s, 1H), 7.94 (d, 2H), 7.50 (d, 2H), 6.92 (d, |
| | | | | | | 1H), 4.40 (d, 2H), 3.79 |
| | | | | | | (s, 3H), 2.40 (s, 3H). |
| 61 | 1-(4-methylbenzene-1- sulfonyl)-N-[(4-methyl- 1,3-thiazol-2-yl)methyl]- 1H-pyrazole-3- carboxamide | 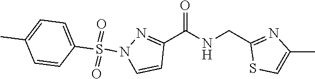 | method B | 377.1 | 2.26 | ¹H NMR (DMSO-d6 400 MHz): δ 9.30- 9.29 (m, 1H), 8.59 (d, 1H), 7.95 (d, 2H), 7.51 (d, 2H), 7.13 (d, 1H), |
| | | | | | | 6.95 (d, 1H), 4.61 (d, |
| | | | | | | 2H), 2.40 (s, 3H), 2.31 |
| | | | | | | (s, 3H). |
| 62 | 1-(4-methylbenzene-1- sulfonyl)-N-[(1-methyl- 1H-pyrazol-4-yl) methyl]-1H-pyrazole- 3-carboxamide | 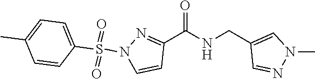 | method B | 359.9 | 1.72 | ¹H NMR (DMSO-d6 400 MHz): δ 8.77- 8.75 (m, 1H), 8.55 (d, 1H), 7.92 (d, 2H), 7.55 (s, 1H), 7.49 (d, 2H), |
| | | | | | | 7.31 (s, 1H), 6.90 (d, |
| | | | | | | 1H), 4.20 (d, 2H), 3.76 |
| | | | | | | (s, 3H), 2.39 (s, 3H). |
| 63 | 1-(4-methylbenzene-1- sulfonyl)-N-[(5- methylpyrazin-2- yl)methyl]-1H-pyrazole- 3-carboxamide | 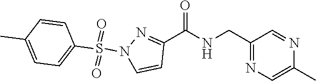 | method B | 372.1 | 2.08 | ¹H NMR (CDCl₃ 400 MHz): δ 8.49 (s, 1H), 8.40 (s, 1H), 8.10 (d, 1H), 7.88 (d, 2H), 7.60 (s, 1H), 7.35 (d, 2H), |
| | | | | | | 6.90 (d, 1H), 4.69 (d, |
| | | | | | | 2H), 2.55 (s, 3H), 2.39 |
| | | | | | | (s, 3H). |
| 64 | 1-(4-methoxyphenyl) sulfonyl-N-(3- pyridylmethyl)pyrazole- 3-carboxamide | 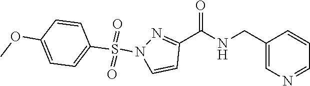 [SMILES: COc1ccc(S(=O)(=O)n2ccc(C(=O)NCc3cccnc3)n2)cc1] | method D | 372.8 | 0.43 | nd |
| 65 | 1-[4-(difluoromethoxy) phenyl]sulfonyl-N-[(5- methylpyrazin-2- yl)methyl]pyrazole-3- carboxamide | 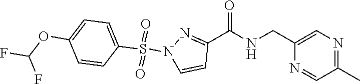 [SMILES: Cc1cnc(CNC(=O)c2ccn(S(=O)(=O)c3ccc(OC(F)F)cc3)n2)cn1] | method D | 423.8 | 0.61 | nd |
| 66 | 1-(4-fluoro-2-methyl- phenyl)sulfonyl-N-[(5- methylpyrazin-2- yl)methyl]pyrazole-3- carboxamide | 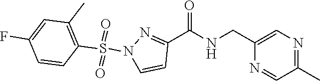 [SMILES: Cc1cnc(CNC(=O)c2ccn(S(=O)(=O)c3ccc(F)cc3C)n2)cn1] | method D | 389.7 | 0.62 | nd |
| 67 | 1-(4-chloro-2-methyl- phenyl)sulfonyl-N-[(5- methylpyrazin-2- yl)methyl]pyrazole-3- carboxamide | 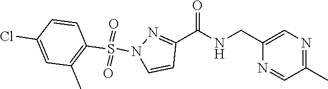 [SMILES: Cc1cnc(CNC(=O)c2ccn(S(=O)(=O)c3ccc(Cl)cc3C)n2)cn1] | method D | 405.7 | 0.68 | nd |
| 68 | 1-(2-fluoro-4-methoxy- phenyl)sulfonyl-N-[(5- methylpyrazin-2- yl)methyl]pyrazole-3- carboxamide | 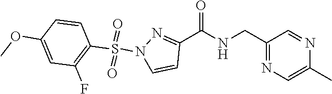 | method D | 405.8 | 0.58 | nd |
| 69 | 1-(3-chloro-4-fluoro- phenyl)sulfonyl-N-[(5- methylpyrazin-2- yl)methyl]pyrazole-3- carboxamide | 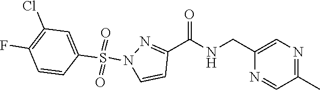 [SMILES: Cc1cnc(CNC(=O)c2ccn(S(=O)(=O)c3ccc(F)c(Cl)c3)n2)cn1] | method D | 409.7 | 0.64 | nd |
| 70 | N-[(5-methylpyrazin-2- yl)methyl]-1-[4- (trifluoromethoxy) phenyl]sulfonyl- pyrazole-3-carboxamide | 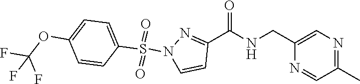 [SMILES: Cc1cnc(CNC(=O)c2ccn(S(=O)(=O)c3ccc(OC(F)(F)F)cc3)n2)cn1] | method D | 441.8 | 0.68 | nd |
| 71 | 1-[4-(difluoromethyl) phenyl]sulfonyl-N-[(5- methylpyrazin-2- yl)methyl]pyrazole-3- carboxamide | 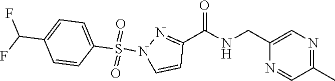 [SMILES: Cc1cnc(CNC(=O)c2ccn(S(=O)(=O)c3ccc(C(F)F)cc3)n2)cn1] | method D | 407.8 | 0.60 | nd |
| 72 | 1-(2,3- dihydrobenzofuran-5- ylsulfonyl)-N-[(5- methylpyrazin-2- yl)methyl]pyrazole-3- carboxamide | 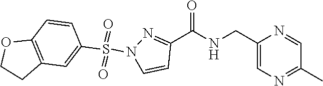 [SMILES: Cc1cnc(CNC(=O)c2ccn(S(=O)(=O)c3ccc4c(c3)CCO4)n2)cn1] | method D | 400 | 0.57 | nd |
| 73 | 1-(4-chloro-2-fluoro- phenyl)sulfonyl-N-[(5- methylpyrazin-2- yl)methyl]pyrazole-3- carboxamide | 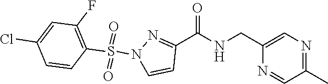 [SMILES: Cc1cnc(CNC(=O)c2ccn(S(=O)(=O)c3ccc(Cl)cc3F)n2)cn1] | method D | 409.9 | 0.63 | nd |
| 74 | 1-(3-fluoro-4-methoxy- phenyl)sulfonyl-N-[(5- methylpyrazin-2- yl)methyl]pyrazole-3- carboxamide | 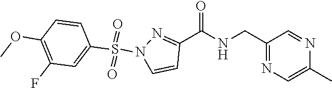 | method D | 405.8 | 0.59 | nd |
| 75 | 1-(3-chlorophenyl) sulfonyl-N- [(5-methylpyrazin-2- yl)methyl]pyrazole-3- carboxamide | 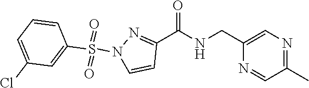 [SMILES: Cc1cnc(CNC(=O)c2ccn(S(=O)(=O)c3cccc(Cl)c3)n2)cn1] | method D | 391.7 | 0.62 | nd |
| 76 | 1-(4-chlorophenyl) sulfonyl-N- [(5-methylpyrazin-2- yl)methyl]pyrazole-3- carboxamide | 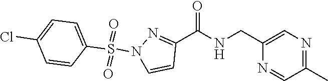 [SMILES: Cc1cnc(CNC(=O)c2ccn(S(=O)(=O)c3ccc(Cl)cc3)n2)cn1] | method D | 391.7 | 0.62 | nd |
| 77 | 1-(2,4-dimethylphenyl) sulfonyl-N-[(5- methylpyrazin-2- yl)methyl]pyrazole-3- carboxamide | 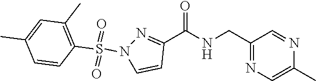 | method D | 385.8 | 0.65 | nd |
| 78 | N-[(5-methylpyrazin-2- yl)methyl]-1-[4- (trifluoromethyl)phenyl] sulfonyl-pyrazole-3- carboxamide | 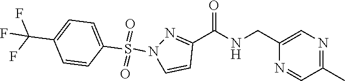 [SMILES: Cc1cnc(CNC(=O)c2ccn(S(=O)(=O)c3ccc(C(F)(F)F)cc3)n2)cn1] | method D | 425.8 | 0.66 | nd |
| 79 | 1-(4- cyclopropylphenyl) sulfonyl-N- [(5-methylpyrazin- 2-yl)methyl]pyrazole-3- carboxamide | 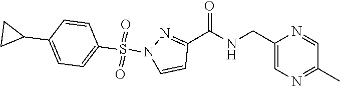 [SMILES: Cc1cnc(CNC(=O)c2ccn(S(=O)(=O)c3ccc(C4CC4)cc3)n2)cn1] | method D | 397.8 | 0.66 | nd |
| 80 | 1-(2-fluoro-4-methyl- phenyl)sulfonyl-N-[(5- methylpyrazin-2- yl)methyl]pyrazole-3- carboxamide | 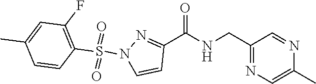 | method D | 389.8 | 0.61 | nd |
| 81 | 1-(4-fluoro- 3,5-dimethyl- phenyl)sulfonyl-N-[(5- methylpyrazin-2- yl)methyl]pyrazole-3- carboxamide | 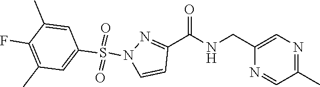 [SMILES: Cc1cnc(CNC(=O)c2ccn(S(=O)(=O)c3cc(C)c(F)c(C)c3)n2)cn1] | method D | 403.8 | 0.69 | nd |
| 82 | 1-(2-chloro-4-methoxy- phenyl)sulfonyl-N-[(5- methylpyrazin-2- yl)methyl]pyrazole-3- carboxamide | 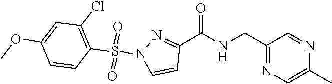 | method D | 421.7 | 0.62 | nd |
| 83 | 1-(4-fluoro- 2,6-dimethyl- phenyl)sulfonyl-N-[(5- methylpyrazin-2- yl)methyl]pyrazole-3- carboxamide | 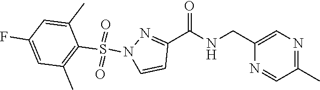 [SMILES: Cc1cnc(CNC(=O)c2ccn(S(=O)(=O)c3c(C)cc(F)cc3C)n2)cn1] | method D | 403.8 | 0.68 | nd |
| 84 | 1-(2-chlorophenyl) sulfonyl-N- [(5-methylpyrazin-2- yl)methyl]pyrazole-3- carboxamide | 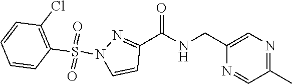 [SMILES: Cc1cnc(CNC(=O)c2ccn(S(=O)(=O)c3ccccc3Cl)n2)cn1] | NO QC | NO QC | NO QC | nd |
| 86 | N-[(3-fluoro-2- pyridyl)methyl]-1-(4- methoxyphenyl) sulfonyl-pyrazole-3- carboxamide | 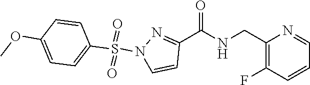 [SMILES: COc1ccc(S(=O)(=O)n2ccc(C(=O)NCc3ncccc3F)n2)cc1] | method D | 390.8 | 0.62 | nd |

Biological Evaluation:

Cell Culture

HEK-293 cells stably expressing hKv3.1b was used for the experiments. Cells were cultured in DMEM medium supplemented with 10% Fetal Bovine Serum, 100 ug/mL Geneticidin and 100 u/mL Penicillin/Streptomycin (all from Gibco). Cells were grown to 80-90% confluency at 37° C. and 5% CO₂. On the day of the experiment the cells were detached from the tissue culture flasks by Detachin and resuspended in serum free medium containing 25 mM HEPES and transferred to the cell hotel of the QPatch. The cells were used for experiments 0-5 hours after detachment.

Electrophysiology

Patch-clamp recordings were performed using the automated recording system QPatch-16x (Sophion Bioscience, Denmark). Cells were centrifuged, SFM removed and the cells were resuspended in extracellular buffer containing (in mM): 145 NaCl, 4 KCl, 1 MgCl₂, 2 CaCl₂, 10 HEPES and 10 glucose (added fresh on the day of experiment); pH 7.4 adjusted with NaOH, 305 mOsm adjusted with sucrose.

Single cell whole-cell recordings were carried out using an intracellular solution containing (in mM): 120 KCl, 32.25/10 KOH/EGTA, 5.374 CaCl₂, 1.75 MgCl₂, 10 HEPES, 4 Na₂ATP (added fresh on the day), pH 7.2 adjusted with KOH, 395 mOsm adjusted with sucrose. Cell membrane potentials were held at −80 mV and currents were evoked by voltage steps (200 ms duration) from −70 mV to +10 mV (in 10 mV increments). Vehicle (0.33% DMSO) or increasing concentration of compound (1) were applied and the voltage protocol was run 3 times (resulting in 3 min cpd incubation time). Five increasing concentrations of compound (1) were applied to each cell.

Leak subtraction protocol was applied at −33% of the sweep amplitude, and serial resistance values were constantly monitored.

Any cell where serial resistance exceeded 25 MOhm, membrane resistance less than 200 MOhm or current size at −10 mV less than 200 pA was eliminated from the subsequent analysis.

Data Analysis

Data analysis was performed using Sophion's QPatch assay software in combination with Microsoft Excel™ (Redmond, Wash., USA).

Current voltage relationships were plotted from the peak current at the individual voltage steps normalized to the vehicle addition at 10 mV. The voltage threshold for channel activation was defined as 5% activation of the peak current at 10 mV in presence of vehicle. The activity of the compounds was described as the ability to shift this current voltage relationship to more hyperpolarized potentials and is given as the maximum absolute shift possible at the tested concentrations (0.37, 1.11, 3.33, 10, 30 μM). Concentration response curves were plotted from the threshold shift at the individual concentrations and were fitted excel fit model 205 sigmodal dose-response model (fit=A+((B−A)/1+((C/x)\hat{ }D)))), where A is the minimum value, B the maximum value, C the EC50 value and D the slope of the curve. The concentration needed to shift the threshold 5 mV was readout from this curve (ECdelta 5 mV).

Compound Effects

In the assay described above, the compounds of the invention had the following biological activity:

| | Max Delta threshold | ECdelta5mV |
|---|---|---|
| Compound | (mV) | (nM) |
| 1 | 23 | 1800 |
| 2 | 27 | 610 |
| 3 | 25 | 1600 |
| 4 | 21 | 2100 |
| 5 | 26 | 1800 |
| 6 | 22 | 1300 |
| 7 | 18 | 2900 |
| 8 | 16 | 3800 |
| 9 | 13 | 3900 |
| 10 | 17 | 3400 |
| 11 | 29 | 2400 |
| 12 | 16 | 3800 |
| 13 | 16 | 4000 |
| 14 | 14 | 4500 |
| 15 | 23 | 2600 |
| 16 | 19 | 1300 |
| 17 | 15 | 3900 |
| 18 | 13 | 7300 |
| 19 | 17 | 2400 |
| 20 | 12 | 3900 |
| 21 | 16 | 3700 |
| 22 | 14 | 3700 |
| 23 | 13 | 5300 |
| 24 | 23 | 2000 |
| 25 | 13 | 6500 |
| 26 | 16 | 2900 |
| 27 | 18 | 3300 |
| 28 | 22 | 2400 |
| 29 | 22 | 3100 |
| 30 | 15 | 3400 |
| 31 | 13 | 4100 |
| 32 | 12 | 4300 |
| 33 | 12 | 2800 |
| 34 | 17 | 2800 |
| 35 | 12 | 4800 |
| 36 | 17 | 1500 |
| 37 | 27 | 2400 |
| 38 | 21 | 1100 |
| 39 | 21 | 1600 |
| 40 | 21 | 2000 |
| 41 | 25 | 1200 |
| 42 | 18 | 2000 |
| 43 | 25 | 1600 |
| 44 | 25 | 1400 |
| 45 | 15 | 2800 |
| 46 | 22 | 1800 |
| 47 | 16 | 3300 |
| 48 | 24 | 1500 |
| 49 | 19 | 2000 |
| 50 | 24 | 1700 |
| 51 | 16 | 3300 |
| 52 | 16 | 1500 |
| 53 | 20 | 1900 |
| 54 | 21 | 2400 |
| 55 | 20 | 1800 |
| 56 | 24 | 1000 |
| 57 | 13 | 4900 |
| 58 | 17 | 2000 |
| 59 | 14 | 3000 |
| 60 | 18 | 2900 |
| 61 | 19 | 2000 |
| 62 | 25 | 1600 |
| 63 | 36 | 1400 |
| 64 | 24 | 1600 |
| 65 | 25 | 3600 |
| 66 | 31 | 2400 |
| 67 | 34 | 1100 |
| 68 | 42 | 2400 |
| 69 | 16 | 4600 |
| 70 | 19 | 4000 |
| 71 | 27 | 3500 |
| 72 | 24 | 2200 |
| 73 | 33 | 2400 |
| 74 | 17 | 3200 |
| 75 | 17 | 3200 |
| 76 | 25 | 1500 |
| 77 | 29 | 1100 |
| 78 | 23 | 3000 |
| 79 | 32 | 990 |
| 80 | 31 | 1500 |
| 81 | 17 | 3000 |
| 82 | 29 | 1600 |
| 83 | 21 | 2700 |
| 84 | 12 | 7700 |
| 86 | 19 | 2400 |

### REFERENCES

Bartos M, Vida I, Jonas P. Synaptic mechanisms of synchronized gamma oscillations in inhibitory interneuron networks. Nat Rev Neurosci. 2007 January; 8(1):45-56. Review.
Chien L Y, Cheng J K, Chu D, Cheng C F, Tsaur M L. Reduced expression of A-type potassium channels in primary sensory neurons induces mechanical hypersensitivity. J Neurosci. 2007 Sep. 12; 27(37):9855-65. PubMed PMID: 17855600.
Chow A, Erisir A, Farb C, Nadal M S, Ozaita A, Lau D, Welker E, Rudy B. K(+) channel expression distinguishes subpopulations of parvalbumin- and somatostatin-containing neocortical interneurons. J Neurosci. 1999 Nov. 1; 19(21):9332-45.
Edden R A, Crocetti D, Zhu H, Gilbert D L, Mostofsky S H. Reduced GABA concentration in attention-deficit/hyperactivity disorder. Arch Gen Psychiatry. 2012 July; 69(7):750-3. doi: 10.1001/archgenpsychiatry.2011.2280.
Foss-Feig J H, Adkinson B D, Ji J L, Yang G, Srihari V H, McPartland J C, Krystal J H, Murray J D, Anticevic A. Searching for Cross-Diagnostic Convergence: Neural Mechanisms Governing Excitation and Inhibition Balance in Schizophrenia and Autism Spectrum Disorders. Biol Psychiatry. 2017 May 15; 81(10):848-861. doi: 10.1016/j.biopsych.2017.03.005. Epub 2017 Mar. 14. Review.
Fuchs T, Jefferson S J, Hooper A, Yee P H, Maguire J, Luscher B. Disinhibition of somatostatin-positive GABAergic interneurons results in an anxiolytic and antidepressant-like brain state. Mol Psychiatry. 2017 June; 22(6):920-930. doi: 10.1038/mp.2016.188. Epub 2016 Nov. 8.
Herrmann C S, Demiralp T. Human EEG gamma oscillations in neuropsychiatric disorders. Clin Neurophysiol. 2005 December; 116(12):2719-33. Epub 2005 Oct. 25. Review.
Kaczmarek L K, Zhang Y. Kv3 Channels: Enablers of Rapid Firing, Neurotransmitter Release, and Neuronal Endurance. Physiol Rev. 2017 Oct. 1; 97(4):1431-1468. doi: 10.1152/physrev.00002.2017. Review.
Klempan T A, Sequeira A, Canetti L, Lalovic A, Ernst C, ffrench-Mullen J, Turecki G. Altered expression of genes involved in ATP biosynthesis and GABAergic neurotransmission in the ventral prefrontal cortex of suicides with and without major depression. Mol Psychiatry. 2009 February; 14(2):175-89. Epub 2007 Oct. 16
Kudo T, Loh D H, Kuljis D, Constance C, Colwell C S. Fast delayed rectifier potassium current: critical for input and output of the circadian system. J Neurosci. 2011 Feb. 23; 31(8):2746-55. doi: 10.1523/JNEUROSCI.5792-10.2011.
Lau D, Vega-Saenz de Miera E C, Contreras D, Ozaita A, Harvey M, Chow A, Noebels J L, Paylor R, Morgan J I, Leonard C S, Rudy B. Impaired fast-spiking, suppressed cortical inhibition, and increased susceptibility to seizures in mice lacking Kv3.2 K⁺ channel proteins. J Neurosci. 2000 Dec. 15; 20(24):9071-85.
Lin L C, Sibille E. Reduced brain somatostatin in mood disorders: a common pathophysiological substrate and drug target? Front Pharmacol. 2013 Sep. 9; 4:110. doi: 10.3389/fphar.2013.00110. Review.
Macica C M, von Hehn C A, Wang L Y, Ho C S, Yokoyama S, Joho R H, Kaczmarek L K. Modulation of the kv3.1b potassium channel isoform adjusts the fidelity of the firing pattern of auditory neurons. J Neurosci. 2003 Feb. 15; 23(4):1133-41.
Muona M, et al. A recurrent de novo mutation in KCNC1 causes progressive myoclonus epilepsy. Nat Genet. 2015 January; 47(1):39-46.
Oliver K L, et al. Myoclonus epilepsy and ataxia due to KCNC1 mutation: Analysis of 20 cases and K(+) channel properties. Ann Neurol. 2017 May; 81(5):677-689. doi: 10.1002/ana.24929
Palop J J, Mucke L. Network abnormalities and interneuron dysfunction in Alzheimer disease. Nat Rev Neurosci. 2016 December; 17(12):777-792. doi: 10.1038/nrn.2016.141. Epub 2016 Nov. 10. Review.
Rudy B, McBain C J. Kv3 channels: voltage-gated K⁺ channels designed for high-frequency repetitive firing. Trends Neurosci. 2001 September; 24(9):517-26. Review.
Straub R E, Lipska B K, Egan M F, Goldberg T E, Callicott J H, Mayhew M B, Vakkalanka R K, Kolachana B S, Kleinman J E, Weinberger D R. Allelic variation in GAD1 (GAD67) is associated with schizophrenia and influences cortical function and gene expression. Mol Psychiatry. 2007 September; 12(9):854-69. Epub 2007 May 1.
Strumbos J G, Brown M R, Kronengold J, Polley D B, Kaczmarek L K. Fragile X mental retardation protein is required for rapid experience-dependent regulation of the potassium channel Kv3.1b. J Neurosci. 2010 Aug. 4; 30(31):10263-71. doi: 10.1523/JNEUROSCI.1125-10.2010.
Tsantoulas C, McMahon S B. Opening paths to novel analgesics: the role of potassium channels in chronic pain. Trends Neurosci. 2014 March; 37(3):146-58. doi: 10.1016/j.tins.2013.12.002. Epub 2014 Jan. 21. Review.
Veit J, Hakim R, Jadi M P, Sejnowski T J, Adesnik H. Cortical gamma band synchronization through somatostatin interneurons. Nat Neurosci. 2017 July; 20(7):951-959. doi: 10.1038/nn.4562. Epub 2017 May 8.
von Hehn C A, Bhattacharjee A, Kaczmarek L K. Loss of Kv3.1 tonotopicity and alterations in cAMP response element-binding protein signaling in central auditory neurons of hearing impaired mice. J Neurosci. 2004 Feb. 25; 24(8):1936-40.
Weiser M, Vega-Saenz de Miera E, Kentros C, Moreno H, Franzen L, Hillman D, Baker H, Rudy B. Differential expression of Shaw-related K⁺ channels in the rat central nervous system. J Neurosci. 1994 March; 14(3 Pt 1):949-72.

1. A compound (I) of Formula I 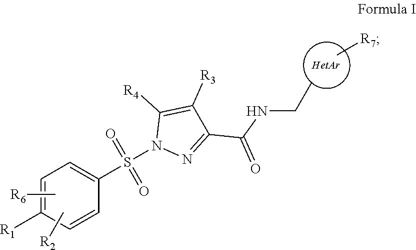
or a pharmaceutically acceptable salt thereof;
wherein:
R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, C₁-C₄ thioalkyl, C₁-C₄ thiofluoroalkyl, and halogen;
R2 and R6 are independently selected from the group consisting of H, C₁-C₄ alkyl, C₁-C₄ alkoxy, and halogen;
R3 is selected from the group consisting of H, fluorine, and C₁-C₄ alkyl;
R4 is selected from the group consisting of H and fluorine;
R7 is selected from the group consisting of H, C₁-C₄ alkyl, halogen, C₁-C₄ alkoxy, C₁-C₄ fluoroalkyl, and C₁-C₄ fluoroalkoxy;
HetAr is selected from the group consisting of 5-membered heteroaryl and 6-membered heteroaryl;
wherein when R1 is C₁-C₄ alkoxy, R1 optionally forms a ring with R2 or R6 when any one of R2 or R6 are C₁-C₄ alkyl.

2. The compound (I) according to claim 1, or a pharmaceutically acceptable salt thereof, wherein:
R1 is selected from the group consisting of H, C₁-C₄ alkyl, C₁-C₄ fluoroalkyl, C₁-C₄ alkoxy, C₁-C₄ fluoroalkoxy, C₃-C₈ cycloalkyl, and halogen;
R2 and R6 are independently selected from the group consisting of H, C₁-C₄ alkyl, and halogen;
R3 is selected from the group consisting of H and C₁-C₄ alkyl;
R4 is hydrogen;
R7 is selected from the group consisting of H, C₁-C₄ alkyl, and halogen;
HetAr is selected from the group consisting of 5-membered heteroaryl and 6-membered heteroaryl; and
wherein when R1 is C₁-C₄ alkoxy, R1 optionally forms a ring with R2 or R6 when any one of R2 or R6 are C₁-C₄ alkyl.

3. The compound (I) according to claim 2, or a pharmaceutically acceptable salt thereof, wherein R1 is selected from the group consisting of hydrogen, methyl, difluoromethyl, trifluoromethyl, trifluoromethoxy, difluoromethoxy, cyclopropyl, fluorine, chlorine, and methoxy.

4. The compound (I) according to claim 2, or a pharmaceutically acceptable salt thereof, wherein R2 and R6 are independently selected from the group consisting of hydrogen, fluorine, chlorine, and methyl.

5. The compound (I) according to claim 2, or a pharmaceutically acceptable salt thereof, wherein R3 is selected from the group consisting of hydrogen and methyl.

6. The compound (I) according to claim 1, or a pharmaceutically acceptable salt thereof, wherein R4 is hydrogen.

7. The compound (I) according to claim 2, or a pharmaceutically acceptable salt thereof, wherein R7 is selected from the group consisting of hydrogen, fluorine, and methyl.

8. The compound (I) according to claim 2, or a pharmaceutically acceptable salt thereof, wherein R1 is methoxy and forms a ring closure with R2 or R6 when R2 or R6 any one of these are methyl.

9. The compound (I) according to claim 2, or a pharmaceutically acceptable salt thereof, wherein HetAr is selected from the group consisting of pyrimidinyl, pyrazinyl, pyrazolyl, pyridyl, oxadiazolyl, isoxazolyl, oxazolyl, thiazolyl, isothiazolyl, imidazolyl, triazolyl, and thiadiazolyl.

10. The compound according to claim 1, or a pharmaceutically acceptable salt thereof, selected from the group consisting of:
1-(4-methoxy-3-methylbenzene-1-sulfonyl)-N-[(5-methylpyrazin-2-yl)methyl]-1H-pyrazole-3-carboxamide;
1-(4-methoxy-2-methylbenzene-1-sulfonyl)-N-[(5-methylpyrazin-2-yl)methyl]-1H-pyrazole-3-carboxamide;
1-(4-methoxy-2-methylbenzene-1-sulfonyl)-N-[(pyrazin-2-yl)methyl]-1H-pyrazole-3-carboxamide;
1-(4-methoxy-3-methylbenzene-1-sulfonyl)-N-[(pyrazin-2-yl)methyl]-1H-pyrazole-3-carboxamide;
1-(4-methoxybenzene-1-sulfonyl)-N-[(5-methylpyrazin-2-yl)methyl]-1H-pyrazole-3-carboxamide;
1-(4-methoxybenzene-1-sulfonyl)-N-[(4-methyl-1,3-thiazol-2-yl)methyl]-1H-pyrazole-3-carboxamide;
1-(4-methoxybenzene-1-sulfonyl)-N-[(pyrimidin-5-yl)methyl]-1H-pyrazole-3-carboxamide;
1-(4-methoxybenzene-1-sulfonyl)-N-[(1,2-oxazol-3-yl)methyl]-1H-pyrazole-3-carboxamide;
1-(4-methoxybenzene-1-sulfonyl)-N-[(1,2-oxazol-4-yl)methyl]-1H-pyrazole-3-carboxamide;
1-(4-methoxybenzene-1-sulfonyl)-N-[(1,2-oxazol-5-yl)methyl]-1H-pyrazole-3-carboxamide;
1-(4-methoxybenzene-1-sulfonyl)-N-[(2-methylpyrimidin-5-yl)methyl]-1H-pyrazole-3-carboxamide;
1-(4-methoxybenzene-1-sulfonyl)-N-[(1,3-oxazol-4-yl)methyl]-1H-pyrazole-3-carboxamide;
1-(4-methoxybenzene-1-sulfonyl)-N-[(1-methyl-1H-1,2,4-triazol-3-yl)methyl]-1H-pyrazole-3-carboxamide;
1-(benzenesulfonyl)-N-[(3-methyl-1,2-oxazol-5-yl)methyl]-1H-pyrazole-3-carboxamide;
1-(4-methylbenzene-1-sulfonyl)-N-[(5-methylpyrimidin-2-yl)methyl]-1H-pyrazole-3-carboxamide;
1-(4-methylbenzene-1-sulfonyl)-N-[(5-methylpyridin-2-yl)methyl]-1H-pyrazole-3-carboxamide;
1-(2-methylbenzene-1-sulfonyl)-N-[(5-methyl-1,3-oxazol-2-yl)methyl]-1H-pyrazole-3-carboxamide;
1-(3-methylbenzene-1-sulfonyl)-N-[(5-methyl-1,3-oxazol-2-yl)methyl]-1H-pyrazole-3-carboxamide;
1-(3-methylbenzene-1-sulfonyl)-N-[(3-methyl-1,2-oxazol-5-yl)methyl]-1H-pyrazole-3-carboxamide;
1-(3-methylbenzene-1-sulfonyl)-N-[(5-methylpyridin-2-yl)methyl]-1H-pyrazole-3-carboxamide;
1-(3-methylbenzene-1-sulfonyl)-N-[(1-methyl-1H-pyrazol-4-yl)methyl]-1H-pyrazole-3-carboxamide;
1-(3-methylbenzene-1-sulfonyl)-N-[(1-methyl-1H-pyrazol-3-yl)methyl]-1H-pyrazole-3-carboxamide;
1-(2-methylbenzene-1-sulfonyl)-N-[(3-methyl-1,2,4-oxadiazol-5-yl)methyl]-1H-pyrazole-3-carboxamide;
1-(4-methylbenzene-1-sulfonyl)-N-[(pyrazin-2-yl)methyl]-1H-pyrazole-3-carboxamide;
1-(2-methylbenzene-1-sulfonyl)-N-[(5-methylpyrimidin-2-yl)methyl]-1H-pyrazole-3-carboxamide;
1-(2-methylbenzene-1-sulfonyl)-N-[(1-methyl-1H-pyrazol-3-yl)methyl]-1H-pyrazole-3-carboxamide;
1-(2-methylbenzene-1-sulfonyl)-N-[(5-methylpyridin-2-yl)methyl]-1H-pyrazole-3-carboxamide;
1-(2-methylbenzene-1-sulfonyl)-N-[(1-methyl-1H-pyrazol-4-yl)methyl]-1H-pyrazole-3-carboxamide;
1-(2-methylbenzene-1-sulfonyl)-N-[(3-methyl-1,2-oxazol-5-yl)methyl]-1H-pyrazole-3-carboxamide;
1-(4-methylbenzene-1-sulfonyl)-N-[(5-methyl-1,3-oxazol-2-yl)methyl]-1H-pyrazole-3-carboxamide;
1-(4-fluorobenzene-1-sulfonyl)-N-[(3-methyl-1,2-oxazol-5-yl)methyl]-1H-pyrazole-3-carboxamide;
4-methyl-1-(4-methylbenzene-1-sulfonyl)-N-[(5-methylpyrazin-2-yl)methyl]-1H-pyrazole-3-carboxamide;
1-(4-methylbenzene-1-sulfonyl)-N-[(1,2-thiazol-4-yl)methyl]-1H-pyrazole-3-carboxamide;
1-(4-fluorobenzene-1-sulfonyl)-N-[(5-methylpyridin-2-yl)methyl]-1H-pyrazole-3-carboxamide;
1-(4-fluorobenzene-1-sulfonyl)-N-[(1-methyl-1H-pyrazol-3-yl)methyl]-1H-pyrazole-3-carboxamide;
1-(4-methoxybenzene-1-sulfonyl)-N-[(5-methyl-1,3-oxazol-2-yl)methyl]-1H-pyrazole-3-carboxamide;
1-(4-methoxybenzene-1-sulfonyl)-N-[(5-methylpyrimidin-2-yl)methyl]-1H-pyrazole-3-carboxamide;
1-(4-methoxybenzene-1-sulfonyl)-N-[(3-methyl-1,2-oxazol-5-yl)methyl]-1H-pyrazole-3-carboxamide;
1-(4-methylbenzene-1-sulfonyl)-N-[(1-methyl-1H-pyrazol-3-yl)methyl]-1H-pyrazole-3-carboxamide;
1-(4-methoxybenzene-1-sulfonyl)-N-[(5-methylpyridin-2-yl)methyl]-1H-pyrazole-3-carboxamide;
1-(4-methoxybenzene-1-sulfonyl)-N-[(1-methyl-1H-pyrazol-4-yl)methyl]-1H-pyrazole-3-carboxamide;
1-(4-methoxybenzene-1-sulfonyl)-N-[(3-methyl-1,2,4-oxadiazol-5-yl)methyl]-1H-pyrazole-3-carboxamide;
1-(4-methoxybenzene-1-sulfonyl)-N-[(pyrazin-2-yl)methyl]-1H-pyrazole-3-carboxamide;
1-(4-methoxybenzene-1-sulfonyl)-N-[(1-methyl-1H-pyrazol-3-yl)methyl]-1H-pyrazole-3-carboxamide;
1-(4-methylbenzene-1-sulfonyl)-N-[(1,3-oxazol-2-yl)methyl]-1H-pyrazole-3-carboxamide;
1-(4-methylbenzene-1-sulfonyl)-N-[(1-methyl-1H-1,2,3-triazol-4-yl)methyl]-1H-pyrazole-3-carboxamide;
1-(4-methylbenzene-1-sulfonyl)-N-[(1,3,4-thiadiazol-2-yl)methyl]-1H-pyrazole-3-carboxamide;
1-(4-methylbenzene-1-sulfonyl)-N-[(3-methyl-1,2,4-oxadiazol-5-yl)methyl]-1H-pyrazole-3-carboxamide;
1-(4-methylbenzene-1-sulfonyl)-N-[(1,3-oxazol-4-yl)methyl]-1H-pyrazole-3-carboxamide;
1-(4-methylbenzene-1-sulfonyl)-N-[(2-methylpyrimidin-5-yl)methyl]-1H-pyrazole-3-carboxamide;
1-(4-methylbenzene-1-sulfonyl)-N-[(1,2-oxazol-4-yl)methyl]-1H-pyrazole-3-carboxamide;
1-(4-methylbenzene-1-sulfonyl)-N-[(1,3-thiazol-4-yl)methyl]-1H-pyrazole-3-carboxamide;
1-(4-methylbenzene-1-sulfonyl)-N-[(1-methyl-1H-imidazol-4-yl)methyl]-1H-pyrazole-3-carboxamide;
1-(4-methylbenzene-1-sulfonyl)-N-[(pyrimidin-5-yl)methyl]-1H-pyrazole-3-carboxamide;
1-(4-methylbenzene-1-sulfonyl)-N-[(1,2-oxazol-5-yl)methyl]-1H-pyrazole-3-carboxamide;
1-(4-methylbenzene-1-sulfonyl)-N-[(3-methyl-1,2-oxazol-5-yl)methyl]-1H-pyrazole-3-carboxamide;
1-(4-methylbenzene-1-sulfonyl)-N-[(1,2,4-oxadiazol-3-yl)methyl]-1H-pyrazole-3-carboxamide;
1-(4-methylbenzene-1-sulfonyl)-N-[(1,2-oxazol-3-yl)methyl]-1H-pyrazole-3-carboxamide;
1-(4-methylbenzene-1-sulfonyl)-N-[(1,3-thiazol-2-yl)methyl]-1H-pyrazole-3-carboxamide;
1-(4-methylbenzene-1-sulfonyl)-N-[(1-methyl-1H-1,2,4-triazol-3-yl)methyl]-1H-pyrazole-3-carboxamide;
1-(4-methylbenzene-1-sulfonyl)-N-[(4-methyl-1,3-thiazol-2-yl)methyl]-1H-pyrazole-3-carboxamide;
1-(4-methylbenzene-1-sulfonyl)-N-[(1-methyl-1H-pyrazol-4-yl)methyl]-1H-pyrazole-3-carboxamide;
1-(4-methylbenzene-1-sulfonyl)-N-[(5-methylpyrazin-2-yl)methyl]-1H-pyrazole-3-carboxamide;
1-(4-methoxyphenyl)sulfonyl-N-(3-pyridylmethyl)pyrazole-3-carboxamide;
1-[4-(difluoromethoxy)phenyl]sulfonyl-N-[(5-methylpyrazin-2-yl)methyl]pyrazole-3-carboxamide;
1-(4-fluoro-2-methyl-phenyl)sulfonyl-N-[(5-methylpyrazin-2-yl)methyl]pyrazole-3-carboxamide;
1-(4-chloro-2-methyl-phenyl)sulfonyl-N-[(5-methylpyrazin-2-yl)methyl]pyrazole-3-carboxamide;
1-(2-fluoro-4-methoxy-phenyl)sulfonyl-N-[(5-methylpyrazin-2-yl)methyl]pyrazole-3-carboxamide;
1-(3-chloro-4-fluoro-phenyl)sulfonyl-N-[(5-methylpyrazin-2-yl)methyl]pyrazole-3-carboxamide;
N-[(5-methylpyrazin-2-yl)methyl]-1-[4-(trifluoromethoxy)phenyl]sulfonyl-pyrazole-3-carboxamide;
1-[4-(difluoromethyl)phenyl]sulfonyl-N-[(5-methylpyrazin-2-yl)methyl]pyrazole-3-carboxamide;
1-(2,3-dihydrobenzofuran-5-ylsulfonyl)-N-[(5-methylpyrazin-2-yl)methyl]pyrazole-3-carboxamide;
1-(4-chloro-2-fluoro-phenyl)sulfonyl-N-[(5-methylpyrazin-2-yl)methyl]pyrazole-3-carboxamide;
1-(3-fluoro-4-methoxy-phenyl)sulfonyl-N-[(5-methylpyrazin-2-yl)methyl]pyrazole-3-carboxamide;
1-(3-chlorophenyl)sulfonyl-N-[(5-methylpyrazin-2-yl)methyl]pyrazole-3-carboxamide;
1-(4-chlorophenyl)sulfonyl-N-[(5-methylpyrazin-2-yl)methyl]pyrazole-3-carboxamide;
1-(2,4-dimethylphenyl)sulfonyl-N-[(5-methylpyrazin-2-yl)methyl]pyrazole-3-carboxamide;
N-[(5-methylpyrazin-2-yl)methyl]-1-[4-(trifluoromethyl)phenyl]sulfonyl-pyrazole-3-carboxamide;
1-(4-cyclopropylphenyl)sulfonyl-N-[(5-methylpyrazin-2-yl)methyl]pyrazole-3-carboxamide;
1-(2-fluoro-4-methyl-phenyl)sulfonyl-N-[(5-methylpyrazin-2-yl)methyl]pyrazole-3-carboxamide;
1-(4-fluoro-3,5-dimethyl-phenyl)sulfonyl-N-[(5-methylpyrazin-2-yl)methyl]pyrazole-3-carboxamide;
1-(2-chloro-4-methoxy-phenyl)sulfonyl-N-[(5-methylpyrazin-2-yl)methyl]pyrazole-3-carboxamide;
1-(4-fluoro-2,6-dimethyl-phenyl)sulfonyl-N-[(5-methylpyrazin-2-yl)methyl]pyrazole-3-carboxamide;
1-(2-chlorophenyl)sulfonyl-N-[(5-methylpyrazin-2-yl)methyl]pyrazole-3-carboxamide; and
N-[(3-fluoro-2-pyridyl)methyl]-1-(4-methoxyphenyl)sulfonyl-pyrazole-3-carboxamide.

11. A pharmaceutical composition comprising a compound according to claim 1, or a pharmaceutically acceptable salt thereof, and one or more pharmaceutically acceptable excipients.

12. A method of treating a disease or disorder comprising administering to a patient in need thereof a therapeutically effective amount of a compound according to claim 1, or a pharmaceutically acceptable salt thereof, or a pharmaceutical composition comprising a compound according to claim 1, or a pharmaceutically acceptable salt thereof, and one or more pharmaceutically acceptable excipients.

13. The method according to claim 12, wherein the disorder is a neurological or psychiatric disorder.

14. (canceled)

15. (canceled)

16. The method according to claim 13, wherein the neurological or psychiatric disorder is selected from the group consisting of epilepsy, schizophrenia, schizophreniform disorder, schizoaffective disorder, cognitive impairment associated with schizophrenia (CIAS), autism spectrum disorder, bipolar disorder, attention-deficit/hyperactivity disorder (ADHD), anxiety-related disorders, depression, cognitive dysfunction, Alzheimer's disease, Fragile X syndrome, chronic pain, hearing loss, sleep and circadian disorders, and sleep disruption.

17. The method according to claim 16, wherein the schizophrenia is the paranoid, disorganized, catatonic, undifferentiated, or residual type.

18. The method according to claim 16, wherein the schizoaffective disorder is the delusional type or the depressive type.
